(19) United States
(12) Patent Application Publication
(10) Pub. No.: US 2025/0127890 A1
(43) Pub. Date: Apr. 24, 2025

(54) COMPOSITIONS AND METHODS FOR TREATING CORONAVIRUS INFECTIOUS DISEASE-19 (COVID-19)

(71) Applicant: THE BOARD OF REGENTS OF THE UNIVERSITY OF OKLAHOMA, Norman, OK (US)

(72) Inventors: Naushad ALI, Norman, OK (US); Mark M. HUYCKE, Fayetteville, AR (US)

(73) Assignee: THE BOARD OF REGENTS OF THE UNIVERSITY OF OKLAHOMA, Norman, OK (US)

(21) Appl. No.: 18/570,121

(22) PCT Filed: Oct. 11, 2022
(86) PCT No.: PCT/US2022/077893

§ 371 Date: Dec. 14, 2023

Related U.S. Application Data
(60) Provisional application No. 63/254,531, filed on Oct. 11, 2021.

(51) Int. Cl. A61K 39/395; A61K 45/06; A61P 31/14; C07K 16/40; C12N 15/113; G01N 33/569

(52) U.S. Cl. CPC A61K 39/3955; A61K 45/06; A61P 31/14; C07K 16/40; C12N 15/1137; C12Y 207/11001; G01N 33/56972

(57) ABSTRACT

Compositions and methods for treating and/or diagnosing a subject having COVID-19. The treatment composition includes an inhibitor of at least one of doublecortin-like kinase 1 (DCLK1, including DCLK1 isoforms 1-4), and doublecortin-like kinase 2 (DCLK2, including DCLK2 isoforms 1-3). The treatment composition may optionally include an inhibitor of SI 00 calcium binding protein A9 (S100A9), calprotectin (S100A8/S100A9 complex), S100A4, Granulocyte-macrophage colony-stimulating factor (GM-CSF), Vascular endothelial growth factor (VEGF), Interleukin-6 (IL-6), or combinations thereof. The subject may also have a chronic liver disease, disorder, or condition. A method of determining if a patient having COVID-19 should be administered a treatment protocol for severe or critical COVID-19.

### CROSS-REFERENCE TO RELATED APPLICATIONS

The present application claims the benefit of U.S. Provisional Patent Application Ser. No. 63/254,531, filed Oct. 11, 2021, the disclosure of which is hereby incorporated by reference in its entirety.

### STATEMENT OF GOVERNMENT SUPPORT

This invention was made with government support under Contract Numbers GM103447 and CA230641 awarded by the National Institutes of Health. The government has certain rights in the invention.

### SEQUENCE LISTING

This application contains a Sequence Listing XML, which has been submitted electronically and is hereby incorporated by reference in its entirety. Said XML Sequence Listing, created on Oct. 5, 2022, is named OKLAP0012WO_ST26.xml and is 5,431 bytes in size.

### BACKGROUND

The Coronavirus infectious disease-19 (COVID-19) pandemic represents the most significant global public health crisis since the influenza outbreak of 1918. Severe acute respiratory syndrome coronavirus-2 (SARS-CoV-2) and variants of concern (e.g., delta, omicron) are causative agents of the current COVID-19 pandemic. Many people who contract COVID-19 are asymptomatic or only present with mild symptoms (80%) and recover. However, in a minority of cases infection can rapidly progress to hypoxia and acute respiratory distress syndrome (ARDS). This is especially true for elderly patients and those with underlying co-morbid medical conditions. Deep immune profiling of COVID-19 patients has revealed heterogenous immune responses. A characteristic cytokine storm has also been noted in severely ill patients. Although lung is the primarily affected organ in severe SARS-CoV-2 infection, tissue- and organo-tropism has been documented. Among comorbid factors for COVID-19, pre-existing liver disease is associated with particularly poor outcomes and high mortality rates. However, the underlying mechanisms for this are poorly understood.

SARS-CoV-2 is a positive-strand RNA β-coronavirus. The viral genome displays extensive homology with SARS-CoV-1 and MERS-CoV. A viral Spike glycoprotein binds to cells via surface angiotensin-converting enzyme 2 (ACE2). Following engagement, Spike is cleaved by a type II transmembrane serine protease and TMPRSS2 to facilitate viral internalization. As a result, ACE2 is also internalized and subsequently downregulated. This leads to a decrease in the conversion of its natural substrate, angiotensin II (AngII), to angiotensin-(1-7). As a result AngII levels increase, and when bound to the AT₁R receptor inflammatory pathways such as nuclear factor (NF)-κB are activated with the subsequent expression of multiple downstream cytokines. However, ACE inhibitors and angiotensin receptor blockers (ARBs) do not show treatment benefits in COVID-19 suggesting other mechanisms help drive inflammation, immune cell dysregulation, and the cytokine storm.

In spite of the approval of and widespread administration of anti-coronavirus vaccines in 2021, COVID-19 continues to be a serious problem due to non-use of the vaccines by much of the population. Thus, therapies for treating COVID-19 are likely to be necessary for the foreseeable future. It is to meeting this need that the present disclosure is directed.

### BRIEF DESCRIPTION OF THE DRAWINGS

This patent or application file contains at least one drawing executed in color. Copies of this patent or patent application publication with color drawings will be provided by the Office upon request and payment of the necessary fee.

FIG. 1A shows H&E staining results representative of autopsied lung (upper panel of 8 images) and liver (lower panel of 8 images) tissues from COVID-19 patients who had chronic liver disease (CLD). Photomicrographs show two separate areas for each individual. Histopathologic interpretations are summarized in Table 1. Magnification: 20×.

FIG. 1B shows H&E staining results representative of autopsied lung (upper panel of 6 images) and liver (lower panel of 6 images) tissues from non-COVID-19 patients with severe lung diseases. Photomicrographs show two separate areas for each individual. Histopathologic interpretations are summarized in Table 1. Magnification: 20×.

FIG. 2A DCLK1 expression and SARS-CoV-2 infection in lung for a representative COVID-19 case and normal control. Lung tissues from one COVID-19 autopsy and from a normal control were immunostained for SARS-CoV-2 Spike (red), ACE2 (green), and DCLK1 (magenta) and imaged by confocal microscopy. Cellular staining patterns are highlighted (yellow boxes; right panels, highlighted). Intracellular Spike is seen at sites of active SARS-CoV-2 infection. Nuclear stain, blue. Scale, 20 μm.

FIG. 2B DCLK1 expression and SARS-CoV-2 infection in liver for a representative COVID-19 case and normal control. Liver tissues from one COVID-19 autopsy and from a normal control were immunostained for SARS-CoV-2 Spike (red), ACE2 (green), and DCLK1 (magenta) and imaged by confocal microscopy. Cellular staining patterns are highlighted (yellow boxes; right panels, highlighted). Intracellular Spike is seen at sites of active SARS-CoV-2 infection. Nuclear stain, blue. Scale, 20 μm.

FIG. 3A1 shows results of confocal microscopy of lung from autopsied subjects with both COVID-19 and CLD (Cases 1-3), and normal case controls (left vertical panel) after co-staining for S100A9 (green), CD206 (red), DCLK1 (cyan), and nucleus (blue); scale, 20 μm. DCLK1- and S100A9-co-expressing immunosuppressive M2 macrophages (CD206⁺) extensively accumulate in the lungs of COVID-19 individuals with CLD.

FIG. 3A2 shows the comparative absence of DCLK1- and S100A9-co-expressing immunosuppressive M2 macrophages (CD206⁺) in the lungs of non-COVID-19 individuals with lung diseases after co-staining for S100A9 (green), CD206 (red), DCLK1 (cyan), and nucleus (blue); scale, 20 μm.

FIG. 3B shows an enlargement of the highlighted boxed yellow areas in case 1 of FIG. 3A1.

FIG. 3C shows the quantitative evaluation of DCLK1⁺S100A9⁺CD206⁺ macrophages from the stained lungs (50 μm²/area, 15 dots represent 3 cases and 5 sites were evaluated for each case).

FIG. 3D1 shows stained liver tissues of corresponding individuals shown in FIG. 3A1. Tissues were co-stained for S100A9 (green), DCLK1 (cyan), CD206 (red), and nucleus (blue). The images were visualized by confocal microscopy. DCLK1- and S100A9-co-expressing immunosuppressive M2 macrophages (CD206⁺) extensively accumulate in the livers of COVID-19 individuals with CLD.

FIG. 3D2 shows the comparative absence of DCLK1- and S100A9-co-expressing immunosuppressive M2 macrophages (CD206⁺) in the livers of non-COVID-19 individuals with lung diseases after co-staining for S100A9 (green), CD206 (red), DCLK1 (cyan), and nucleus (blue); scale, 20 μm. The images were visualized by confocal microscopy.

FIG. 3E shows an enlargement of the highlighted boxed yellow areas in case 1 of FIG. 3D1. Triple-positive (DCLK1+S100A9+CD206⁺) M2 macrophages accumulated in the liver. Magnification, 60×.

FIG. 3F shows the quantitative evaluation of DCLK1⁺S100A9⁺CD206⁺ macrophages from the stained livers (50 μm²/area), 4 sites in the stained slides were evaluated for each case. Twelve dots (e.g., COVID-19 or Non-COVID-19) represent 3 cases in each group as shown in FIGS. 3D1/3D2. For two normal cases, 8 dots represent 4 sites for each one on the stained slides.

FIG. 4A1 shows flow cytometry of PBMCs from normal adults for DCLK1, S100A9, and M1 (CD86) and M2 (CD206) macrophage markers. Cells were stained with antibody conjugates or corresponding isotype control IgG-conjugates (negative controls used for gating).

FIG. 4A2 shows flow cytometry of PBMCs from Covid-19 patients for DCLK1, S100A9, and M1 (CD86) and M2 (CD206) macrophage markers. Cells were stained with antibody conjugates or corresponding isotype control IgG-conjugates (negative controls used for gating).

FIG. 4B shows flow cytometry of PBMCs analyzed for DCLK1, S100A9, and M1 (CD86) and M2 (CD206) macrophage markers. Cells were stained with antibody conjugates or corresponding isotype control IgG-conjugates (negative controls used for gating). The percent of DCLK1⁺S100A9⁺ cells in PBMC populations from seven severe/critical and four mild/moderate COVID-19 patients and three normal healthy adults are shown.

FIG. 4C shows results for sera from 3 normal, 6 mild/moderate COVID-19, and 11 severe/critical COVID-19 patients that were analyzed for various cytokines. Normal is lefthand bar, mild/moderate is center bar, righthand bar is severe/critical. Cytokine levels were arbitrarily set at 100 for normal and compared with patients' sera. COVID-19 patients exhibit high level DCLK1 and S100A9 co-expressing mononuclear cells in the blood that correlate with severity of COVID-19. Cells were stained with antibody conjugates or corresponding isotype control IgG-conjugates (negative controls used for gating).

FIG. 5A. Calu3 lung cells infected with SARS-CoV-2 (multiplicity of infection=1) were treated with DCLK1 kinase inhibitor (DCLK1-IN-1). Spike, nucleocapsid, and DCLK1 were detected by Western blot with replicates performed. DCLK1 kinase facilitates production of infectious SARS-CoV-2 particles and inflammatory signaling.

FIG. 5B. Titers of SARS-CoV-2 virions in spent media at 0, 5, and 10 μM DCLK1-INH-1 were assessed by TCID₅₀. P-values: P<0.0005 (***), P<0.00005 (****).

FIG. 5C compares Spike after treatment of infected Calu3 cells with 5 μM of DCLK1-IN-1 (lane 3) or DCLK1-NEG (small molecule negative control, lane 4).

FIG. 5D shows results of confocal microscopy for DCLK1 (green) and Spike (red) in uninfected Calu3 cells (upper), infected but untreated cells (middle), and inhibitor-treated cells (lower).

FIG. 5E shows magnified cells show patterns of Spike (red) and DCLK1 (green) expression in uninfected, infected, and inhibitor-treated Calu3 cells with infection.

FIG. 5F1 shows Western blots of total cell lysates from uninfected (lane 1) and infected (lane 2) Calu3 cells. Infected cells were treated with DCLK1-IN-1 (5 μM, lane 3), tasquinimod (TasQ, 10 μM, lane 4), or both (lane 5).

FIG. 5F2 shows Western blots of total cell lysates from uninfected (lane 1) and infected (lane 2) Calu3 cells. Infected cells were treated with DCLK1-IN-1 (5 μM, lane 3), tasquinimod (TasQ, 10 μM, lane 4), or both (lane 5).

FIG. 5G. Spent media (from above experiments) were assayed for IFNβ (indicates antiviral responses) for infected Calu3 cells (bars 2-5) compared to uninfected controls (left-most bar), 1. Infected cells were treated with DCLK1-IN-1 (blue) or tasquinimod (green) or both (cyan). Data are mean±SEM.

FIG. 6A1 shows procedures for how normal human PBMCs were cultured with uninfected or SARS-CoV-2-infected Calu3 cells for 48 h in a dual-chamber culture system. PBMCs were cultured in upper inserts while uninfected or infected Calu3 cells were in lower chambers. PBMCs were analyzed by multicolor flow cytometry after staining with antibody-fluorophore conjugates. Respective isotype IgG-fluorophore conjugates were used as negative controls for gating (not shown). Live cells with large (SSC-high, black circles) or low (SSC-low, red circles) granular morphology were analyzed.

FIGS. 6A2, 6A3, and 6A4 show that SARS-CoV-2-infected cells induce DCLK1⁺S100A9⁺ monocytes and M1-like macrophages in normal human PBMCs. Cells with large granular morphology were predominantly DCLK1⁺S100A9⁺ (middle panels). Low granularity cells (predominantly monocytes and lymphocytes) were analyzed for CD86⁺ (M1-like) and CD206⁺ (M2-like) polarized macrophages (right panel) with quadrants defined by isotype-IgG-fluorophore conjugates.

FIGS. 6B1, 6B2, and 6B3 show normal human PBMCs cocultured with infected or uninfected Calu3 cells in the same chamber (mixed cocultures) analyzed by flow cytometry as described above. Live large granular cells (SSC-high, black circles, left panels) were predominantly DCLK1⁺S100A9⁺ (middle panels). Low granularity cells (SSC-low, red circles, left panels) represent lymphocytes and monocytes. Analysis of these cells showed an increase (approximately 3-fold) in the proportion of CD86⁺ (M1-like) cells in the mixed cocultures (right bottom panel) as compared to the uninfected control (middle right panel).

FIG. 6C. Spent media from dual-chamber cocultures were analyzed for cytokines, chemokines, and growth factors using a Luminex Assay Kit; samples assayed in duplicate with SEM shown.

FIG. 6D. Spent media from mixed cocultures were analyzed for cytokines, chemokines, and growth factors using a Luminex Assay Kit; samples assayed in duplicate with SEM shown.

FIG. 6E shows a Western blot for the total lysates of infected and uninfected mixed cocultures for probing activated NF-κB (p-NF-κB(65)^{Ser536} and total NF-κB.

FIG. 7A shows that DCLK1-mediated upregulation of SARS-CoV-2 production in the liver promotes viremia, inflammatory response, and immune dysfunction. Primary human hepatocytes (PHHs were cultured on a thin layer of Matrigel for 24 h and infected with SARS-CoV-2. After 72 h, cells were imaged by confocal microscopy for ACE2 (green) and Spike (red). Highlighted (yellow box) cells show a pattern of cell membrane and intracellular ACE2 expression in the uninfected cells (upper panel). Lower panel, infected cells.

FIG. 7B shows confocal microscopy of uninfected (upper panel) and infected (lower panel) PHHs for S100A9 (green) and DCLK1 (red). Highlighted panel (right) shows DCLK1⁺S100A9⁺ PHHs after the viral infection. Intense staining of cellular cytoplasm with Dapi (blue) in infected PHHs is likely due to viral RNA (bottom panel), which is not present in uninfected cells (upper panel).

FIG. 7C shows Western blot of hepatoma Huh7 cell lysates (lane 1, uninfected; lane 2, infected).

FIG. 7D shows Western blots of mixed cocultures of normal human PBMCs with uninfected (lane 1) or SARS-CoV-2-infected (lane 2) Huh7 cells.

FIG. 7E shows Western blots of mixed cocultures of normal human PBMCs with uninfected (lane 1) or SARS-CoV-2-infected (lane 2) Huh7 cells. SARS-CoV-2 downregulates NLRP3 and enhances intracellular accumulation of unprocessed caspase 1 and IL-1β at early stage of infection (48 hrs of infection) in mixed cocultures. Western blots of total lysates from infected or uninfected cultures of Huh7 cells (lanes 1 and 2). Western bot for mixed cocultures of PBMCs and infected (lane 4) or uninfected (lanes 3) Huh7 cells.

FIG. 7F shows that DCLK1 enhances SARS-CoV-2 production. Huh7, Huh7-RFP (control), and Huh7-RFP-DCLK1 (overexpressing recombinant human DCLK1) cells were infected with SARS-CoV-2 and infectious viral particles in spent media assayed at 24, 48, 72, and 96 h post-infection by TCID₅₀ (P=0.002).

FIG. 7G shows cell survival assay for Huh7-RFP-DCLK1 cells at 24, 48, and 72 hrs of SARS-CoV-2 infection. Uninfected cells at each time-point (not shown) is set as 100% (control).

FIG. 7H shows Western blots of infected Huh7 (lane 4), Huh7-RFP (lane 5), and Huh7-RFP-DCLK1 (lane 6) cells for expression of Spike and nucleocapsid proteins at 48 h.

FIG. 8A shows DCLK1-IN-1 inhibits viral replication and viral-induced inflammatory cytokines in DCLK1-overexpressing cells. Mixed cocultures of normal human PBMCs with SARS-CoV-2-infected Huh7-RFP-DCLK1 cells were treated with 5 μM of DCLK1-IN-1 (Set 2, lane 2) for 48 h. Similar untreated mixed cultures were used as a positive control (lane 1) and other controls as indicated (Set 1, lanes 6-7). Culture lysates without infection show no Spike (lanes 3-7). Set 1, PBMC cultures (lane 6) were exposed to similar amounts of SARS-CoV-2 (lane 7) as in lanes 1 and 2.

FIG. 8B shows results of media supernatants of cultures (Set 1) that were assayed using multiplexed cytokine assay kit. Marker expression in untreated spent media was calibrated at 100% and compared to inhibitor-treated samples in Set 2.

FIG. 8C shows results of media supernatants of cultures (Set 2) that were assayed using multiplexed cytokine assay kit. Marker expression in untreated spent media was calibrated at 100%.

FIG. 9 shows potential (but non-limiting) mechanisms for DCLK1-regulated COVID-19 severity in patients with CLD. SARS-CoV-2 infection of Type II pneumocytes results in β-catenin(p65) activation leading to the transcriptional activation of the DCLK1. This results in the expression of large isoform of DCLK1 mRNA, which can express full-length protein (p82) or smaller isoforms by alternatively spliced mRNA or proteolytic cleavage of DCLK1(p82). Viral infection of these cells also triggers DCLK1-dependent activation of inflammatory cytokines (TNF-α, IL-1β and IFNγ). High level expression of DCLK1 and S100A9 in CLD confers increased virus production and pathogenesis. Circulating viruses infect ACE2- and DCLK1-expressing hepatocytes in COVID-19 for individuals with CLD but not in individuals with healthy livers where these proteins are not expressed. The infected pneumocytes and hepatocytes in CLD patients may produce additive or synergistic effects on viral load and inflammatory responses with polarization of macrophages into pro-inflammatory M1 and immunosuppressive M2 (DCLK1+S100A9+CD206+) phenotypes and by secreting cytokines and colony stimulating factors (GM-CSF, MCSF). M2 macrophages produce IL-10 resulting in suppression of innate immunity. Mechanistically, DCLK1 upregulates S100A9, which can bind to TLR4 and receptors for advanced glycation products to increase NF-κB signaling in immune cells. DCLK1 upregulates S100A9, which can bind to TLR4 and receptors for advanced glycation products to increase NF-κB signaling in immune cells.

FIG. 10A shows that DCLK1 kinase inhibitor (DCLK1-IN-1) normalizes the proteome profile of lung cells (Calu3) infected with SARS-CoV-2. Cell lysates from uninfected (treatment S1), SARS-CoV-2 infected (treatment S2), SARS-CoV-2 infected and treated with vehicle (DMSO, treatment S3), and SARS-CoV-2 infected and treated with DCLK1-IN-1 (treatment S4) were subjected to proteomic analysis. Each condition was performed in triplicate. Principal component analysis (PCA) of total protein abundance for each sample showed close clustering (peak area) for S1 and S4 compared to S2 and S3.

FIG. 10B shows a heat map clustering for differential protein abundance of FIG. 10A.

FIG. 10C shows a heat maps show proteins induced by SARS-CoV-2 (red, S2 and S3) that were normalized by DCLK1-IN-1 (S4 vs. S1, green).

FIG. 10D shows a Volcano plot show significantly increased (dark red circle) and decreased (dark green circle) proteins in S2 compared to S1.

FIGS. 10E-F. A Venn diagram showing 8 selected proteins induced by infection and normalized by inhibitor treatment.

FIG. 10G is a Western blot validating results for GSPT2, WDR75, and SAMD4B proteins.

FIG. 11 shows that certain structural and accessory proteins (spike, membrane protein, nucleoprotein, ORF7a, ORF8, and ORF9b) of SARS-CoV-2 are downregulated by DCLK1-IN-1. S1=uninfected, S2=infected, S3=infected+vehicle, S4=infected+DCLK1-IN-1. P values: ≤0.01 (**), ≤0.001 (***), ≤0.0001 (****), >0.05 (ns, not significant).

FIG. 12A shows certain DCLK1 kinase inhibitor blocks phosphorylation of SR-rich regulatory region of SARS-CoV-2 nucleocapsid protein required for viral transcription-replication. Phosphoserine residues are downregulated by DCLK1-IN-1 (right-most bars in each graph). Peptides sequences are MSDNGPQNQ (SEQ ID NO:1), GFYAEGSRGGSQASSR (SEQ ID NO:2), NSTPGSSR (SEQ ID NO:3), GGSQASSRSSSR (SEQ ID NO:4).

FIG. 12B shows an SR-rich sequence GSRGGSQASSRSSSRSRNSSRNSTPGSSR (SEQ ID NO:5) of the nucleocapsid protein. Red stars show DCLK1-IN-1 downregulation of phosphorylation at five serine residues.

FIG. 12C shows that DCLK1-IN-1 inhibits Omicron virus production in the culture supernatant of infected Calu3. TCID50 data for DMSO (vehicle control) was set at 100 percent and compared with the inhibitor treatments.

FIG. 13A shows that SARS-COV-2-induced DCLK1 and other host proteins in the infected lungs. Western blot of the infected (Group 1, S1-S6) and uninfected Group 4 (U1-U6) lung lysates of K18-hACE2 mice. Each group had 6 (3 M and 3 F) mice.

FIG. 13B shows quantitative evaluation of band intensities for each protein band shown in FIG. 13A.

FIG. 13C shows quantitative evaluation of band intensities for each protein band shown in FIG. 13A.

FIG. 14 shows that DCLK1-IN-1 inhibits SARS-CoV-2 production in a COVID-19 murine model. A. H&E-stained lung tissue from treated (2 mice shown) and untreated controls. B. Western blot for Spike and nucleocapsid in the lungs of two mice from each group.

FIG. 15 shows that SARS-COV-2 infection of K18-hACE2 mice results in extensive expression of Dclk1 and S100A9 in lung. (A) Confocal microscopy images of virus infected (top panel) and uninfected (bottom panel) mouse lung tissues that are stained for human ACE2 (green), Spike (red), Dclk1 (cyan), S100A9 (magenta), and nucleus (blue). DCLK1-IN-1 and vehicle treated stained lung tissues are shown in middle panels. (B) Infected cells (Spike⁺, red) are highlighted for the co-expression of Dclk1 and S100A9.

FIG. 16 shows phosphorylation of DCLK1 in Vero cells infected with SARS-CoV2. In two separate phosphoproteomic studies, SARS-CoV-2 infection was associated with the phosphorylation of DCLK1. Sites marked in bold were the phosphorylated sites identified in these studies. Starred sites show additional infection-specific phosphorylation sites we identified using bioinformatic tools.

FIG. 17 shows micrographic images that demonstrate that hepatocellular carcinoma (HCC) contains cells co-expressing Dclk1 and PD-L1. Liver tissue from Dclk1^{f/f} (control) and HSD-KO at the HCC stage were co-stained for DCLK1 (red) and PD-L1 (green) and imaged by confocal microscopy. Blue, Dapi (nuclear stain). Arrows, indicate highlighted areas presented in the far-right panel.

The following abbreviations may be used herein:
SARS-CoV-2 (severe acute respiratory syndrome coronavirus-2),
COVID-19 (coronavirus infectious disease-19),
DCLK1 (doublecortin-like kinase 1),
DCLK2 (doublecortin-like kinase 2),
PBMCs (peripheral blood mononuclear cells),
IL (interleukin),
IL-1β (interleukin-1 beta),
OSCTR (Oklahoma Shared Clinical and Translation Resources)
OUHSC (Oklahoma University Health Sciences Center)
RFP (red fluorescence protein),
ACE2 (angiotensin-converting enzyme 2),
CD (cluster of differentiation),
CLD (chronic liver disease),
TLR (Toll-like receptor),
TNF-α (tumor necrosis factor-alpha),
IFN (interferon),
IFNα (interferon alpha),
IFNβ (interferon beta),
IFNγ (interferon gamma),
NF-κB (nuclear factor-KB),
PHH (primary human hepatocyte),
CCL2 (C-C Motif Chemokine Ligand 2),
M-CSF (Macrophage colony-stimulating factor),
GM-CSF (Granulocyte-macrophage colony-stimulating factor),
VEGF (Vascular endothelial growth factor),
S100A8 (S100 calcium binding protein A8),
S100A9 (S100 calcium binding protein A9),
NTD (N-terminal domain),
CTD (C-terminal domain).

### DETAILED DESCRIPTION

The present disclosure is directed to compositions and methods for treating and/or diagnosing a subject having COVID-19. In at least one non-limiting embodiment, the composition used in the treatment comprises an inhibitor of at least one of DCLK1 and DCLK2, including isoforms thereof (e.g., DCLK1 isoforms 1-4 and DCLK2 isoforms 1-3). In at least one non-limiting embodiment, the composition used in the treatment comprises an inhibitor of DCLK1 and/or DCLK2, including isoforms thereof (e.g., DCLK1 isoforms 1-4 and DCLK2 isoforms 1-3), and an inhibitor of S100 calcium binding protein A9 (S100A9) and/or calprotectin (S100A8/S100A9 complex). In at least one non-limiting embodiment, the method of treating the subject for COVID-19 includes administering to the subject an inhibitor of DCLK1 and/or DCLK2, including isoforms thereof (e.g., DCLK1 isoforms 1-4 and DCLK2 isoforms 1-3), and optionally an inhibitor of S100A9 and/or calprotectin (S100A8/S100A9 complex). In certain embodiments the subject may also have a liver disorder, disease, or condition. Non-limiting examples of such inhibitors are shown below.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. All of the compounds, compositions, and methods and application and uses thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, patent application publications, and non-patent publications, including published articles, which are identified, listed, or mentioned in the specification or referenced in any portion of this application are herein expressly incorporated by reference in their entirety to the same extent as if each individual patent, patent application publication, and non-patent publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” used to indicate that a value includes the inherent variation of error for the composition, the method used to administer the composition, or the variation that exists among the study subjects. “About” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

By “biologically active” is meant the ability to modify the physiological system of an organism without reference to how the active agent has its physiological effects.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals or subjects within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art. An active agent is a compound or other treatment modality (e.g., a DCLK1 inhibitor) which has a therapeutic benefit in accordance with the present disclosure.

The term “effective amount” refers to an amount of an active agent which is sufficient to exhibit a detectable therapeutic or treatment effect in a subject without excessive adverse side effects (such as substantial toxicity, irritation, and allergic response) commensurate with a reasonable benefit/risk ratio when used in the manner of the present disclosure. The effective amount for a subject will depend upon the subject's type, size, and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition, disease, or symptom thereof. A detectable or measurable improvement includes a subjective or objective decrease, reduction, inhibition, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a disease or condition, or consequences of the disease or condition in a subject.

Where used herein in relation to COVID-19 or a positive SARS-CoV-2 test in a subject, the terms “asymptomatic” or “presymptomatic” refer to a subject who has no symptoms of COVID-19; the term “mild” refers to a subject who has mild symptoms such as fever, cough, or change in taste or smell, and who has no dyspnea (difficult or labored breathing); the term “moderate” refers to a subject who has clinical or radiographic evidence of lower respiratory tract disease and has an oxygen saturation level≥94%; the term “severe” refers to a subject who has an oxygen saturation level<94%, a respiratory rate≥30 breaths/minute, and lung infiltates>50%; and the term “critical” refers to a subject who has respiratory failure, shock, and multiorgan dysfunction or failure.

A decrease or reduction in worsening, such as stabilizing the condition or disease, is also a successful treatment outcome. A therapeutic benefit therefore need not be complete ablation or reversal of the disease or condition, or any one, most, or all adverse symptoms, complications, consequences, or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

In certain embodiments, the subject to be treated also has a chronic liver disease (CLD), such as but not limited to, a fatty liver disease, non-alcoholic fatty liver disease (NAFLD), non-alcoholic steatohepatitis (NASH), cirrhosis, fatty liver disease resulting from hepatitis, fatty liver disease resulting from obesity, fatty liver disease resulting from diabetes, fatty liver disease resulting from insulin resistance, fatty liver disease resulting from hypertriglyceridemia, abetalipoproteinemia, glycogen storage diseases, Wolmans disease, and/or acute fatty liver of pregnancy.

In certain embodiments, the DCLK inhibitor compound described herein is a selective inhibitor of DCLK1, or a selective inhibitor of DCLK2. In certain embodiments, the DCLK inhibitor compound described herein is a dual inhibitor of both DCLK1 and DCLK2.

In certain embodiments, the subject may be administered an additional therapeutic agent, such as but not limited to an anti-inflammatory agent and/or a therapeutic agent for a liver disease. The DCLK inhibitor compound and the additional therapeutic agent may be administered simultaneously, within the same or different compositions, or may be administered sequentially. For example, the DCLK inhibitor compound may be administered first and the additional therapeutic agent administered second. Or, the DCLK inhibitor compound may be administered after the additional therapeutic agent is administered.

In certain embodiments, the treatment of SARS-CoV-2, COVID-19, or any associated condition may comprise but is not limited to administration of: a DCLK1 inhibitor; a DCLK2 inhibitor; a DCLK1 inhibitor plus a DCLK2 inhibitor; a DCLK1 inhibitor plus an S100A9 inhibitor; a DCLK2 inhibitor plus an S100A9 inhibitor; a DCLK1 inhibitor plus a DCLK2 inhibitor plus an S100A9 inhibitor; a DCLK1 inhibitor plus a calprotectin inhibitor; a DCLK2 inhibitor plus a calprotectin inhibitor; a DCLK1 inhibitor plus a DCLK2 inhibitor plus a calprotectin inhibitor; a DCLK1 inhibitor plus an S100A4 inhibitor; a DCLK2 inhibitor plus an S100A4 inhibitor; a DCLK1 inhibitor plus a DCLK2 inhibitor plus an S100A4 inhibitor; a DCLK1 inhibitor plus a GM-CSF inhibitor; a DCLK2 inhibitor plus a GM-CSF inhibitor; a DCLK1 inhibitor plus a DCLK2 inhibitor plus a GM-CSF inhibitor; a DCLK1 inhibitor plus a VEGF inhibitor; a DCLK2 inhibitor plus a VEGF inhibitor; a DCLK1 inhibitor plus a DCLK2 inhibitor plus a VEGF inhibitor; a DCLK1 inhibitor plus an IL-6 inhibitor; a DCLK2 inhibitor plus an IL-6 inhibitor; or a DCLK1 inhibitor plus a DCLK2 inhibitor plus an IL-6 inhibitor.

In certain embodiments, the treatment may comprise but is not limited to administration of a DCLK1 inhibitor selected from an antibody, an RNA, and a small molecule; a DCLK2 inhibitor selected from an antibody, an RNA, and a small molecule; and a DCLK1 inhibitor selected from an antibody, an RNA, and a small molecule plus a DCLK2 inhibitor selected from an antibody, an RNA, and a small molecule.

In certain embodiments, the treatment may comprise but is not limited to administration of a DCLK1 inhibitor selected from an antibody, an RNA, and a small molecule plus an S100A9 inhibitor selected from an antibody, an RNA, and a small molecule; a DCLK2 inhibitor selected from an antibody, an RNA, and a small molecule plus an S100A9 inhibitor selected from an antibody, an RNA, and a small molecule; and a DCLK1 inhibitor selected from an antibody, an RNA, and a small molecule plus a DCLK2 inhibitor selected from an antibody, an RNA, and a small molecule plus an S100A9 inhibitor selected from an antibody, an RNA, and a small molecule.

In certain embodiments, the treatment may comprise but is not limited to administration of a DCLK1 inhibitor selected from an antibody, an RNA, and a small molecule plus a calprotectin inhibitor selected from an antibody, an RNA, and a small molecule; a DCLK2 inhibitor selected from an antibody, an RNA, and a small molecule plus a calprotectin inhibitor selected from an antibody, an RNA, and a small molecule; and a DCLK1 inhibitor selected from an antibody, an RNA, and a small molecule plus a DCLK2 inhibitor selected from an antibody, an RNA, and a small molecule plus a calprotectin inhibitor selected from an antibody, an RNA, and a small molecule.

In certain embodiments, the treatment may comprise but is not limited to administration of a DCLK1 inhibitor selected from an antibody, an RNA, and a small molecule plus an S100A4 inhibitor selected from an antibody, an RNA, and a small molecule; a DCLK2 inhibitor selected from an antibody, an RNA, and a small molecule plus an S100A4 inhibitor selected from an antibody, an RNA, and a small molecule; and a DCLK1 inhibitor selected from an antibody, an RNA, and a small molecule plus a DCLK2 inhibitor selected from an antibody, an RNA, and a small molecule plus an S100A4 inhibitor selected from an antibody, an RNA, and a small molecule.

In certain embodiments, the treatment may comprise but is not limited to administration of a DCLK1 inhibitor selected from an antibody, an RNA, and a small molecule plus a GM-CSF inhibitor selected from an antibody, an RNA, and a small molecule; a DCLK2 inhibitor selected from an antibody, an RNA, and a small molecule plus a GM-CSF inhibitor selected from an antibody, an RNA, and a small molecule; and a DCLK1 inhibitor selected from an antibody, an RNA, and a small molecule plus a DCLK2 inhibitor selected from an antibody, an RNA, and a small molecule plus a GM-CSF inhibitor selected from an antibody, an RNA, and a small molecule.

In certain embodiments, the treatment may comprise but is not limited to administration of a DCLK1 inhibitor selected from an antibody, an RNA, and a small molecule plus a VEGF inhibitor selected from an antibody, an RNA, and a small molecule; a DCLK2 inhibitor selected from an antibody, an RNA, and a small molecule plus a VEGF inhibitor selected from an antibody, an RNA, and a small molecule; and a DCLK1 inhibitor selected from an antibody, an RNA, and a small molecule plus a DCLK2 inhibitor selected from an antibody, an RNA, and a small molecule plus a VEGF inhibitor selected from an antibody, an RNA, and a small molecule.

In certain embodiments, the treatment may comprise but is not limited to administration of a DCLK1 inhibitor selected from an antibody, an RNA, and a small molecule plus an IL-6 inhibitor selected from an antibody, an RNA, and a small molecule; a DCLK2 inhibitor selected from an antibody, an RNA, and a small molecule plus an IL-6 inhibitor selected from an antibody, an RNA, and a small molecule; and a DCLK1 inhibitor selected from an antibody, an RNA, and a small molecule plus a DCLK2 inhibitor selected from an antibody, an RNA, and a small molecule plus an IL-6 inhibitor selected from an antibody, an RNA, and a small molecule.

In certain embodiments, the treatment may comprise but is not limited to administration of a DCLK1 inhibitor plus an S100A9 inhibitor plus a GM-CSF inhibitor; a DCLK2 inhibitor plus an S100A9 inhibitor plus a GM-CSF inhibitor; and a DCLK1 inhibitor plus a DCLK2 inhibitor plus an S100A9 inhibitor plus a GM-CSF inhibitor.

In certain embodiments, the treatment may comprise but is not limited to administration of a DCLK1 inhibitor plus an S100A9 inhibitor plus a calprotectin inhibitor; a DCLK2 inhibitor plus an S100A9 inhibitor plus a calprotectin inhibitor; and a DCLK1 inhibitor plus a DCLK2 inhibitor plus an S100A9 inhibitor plus a calprotectin inhibitor.

In certain embodiments, the treatment may comprise but is not limited to administration of a DCLK1 inhibitor plus an S100A9 inhibitor plus an S100A4 inhibitor; a DCLK2 inhibitor plus an S100A9 inhibitor plus an S100A4 inhibitor; and a DCLK1 inhibitor plus a DCLK2 inhibitor plus an S100A9 inhibitor plus an S100A4 inhibitor.

In certain embodiments, the treatment may comprise but is not limited to administration of a DCLK1 inhibitor plus an S100A9 inhibitor plus a VEGF inhibitor; a DCLK2 inhibitor plus an S100A9 inhibitor plus a VEGF inhibitor; and a DCLK1 inhibitor plus a DCLK2 inhibitor plus an S100A9 inhibitor plus a VEGF inhibitor.

In certain embodiments, the treatment may comprise but is not limited to administration of a DCLK1 inhibitor plus an S100A9 inhibitor plus an IL-6 inhibitor; a DCLK2 inhibitor plus an S100A9 inhibitor plus an IL-6 inhibitor; and a DCLK1 inhibitor plus a DCLK2 inhibitor plus an S100A9 inhibitor plus an IL-6 inhibitor.

In certain embodiments, the treatment may comprise but is not limited to administration of a DCLK1 inhibitor plus a GM-CSF inhibitor plus a calprotectin inhibitor; a DCLK2 inhibitor plus a GM-CSF inhibitor plus a calprotectin inhibitor; and a DCLK1 inhibitor plus a DCLK2 inhibitor plus a GM-CSF inhibitor plus a calprotectin inhibitor.

In certain embodiments, the treatment may comprise but is not limited to administration of a DCLK1 inhibitor plus a GM-CSF inhibitor plus an S100A4 inhibitor; a DCLK2 inhibitor plus a GM-CSF inhibitor plus an S100A4 inhibitor; and a DCLK1 inhibitor plus a DCLK2 inhibitor plus a GM-CSF inhibitor plus an S100A4 inhibitor.

In certain embodiments, the treatment may comprise but is not limited to administration of a DCLK1 inhibitor plus a GM-CSF inhibitor plus a VEGF inhibitor; a DCLK2 inhibitor plus a GM-CSF inhibitor plus a VEGF inhibitor; and a DCLK1 inhibitor plus a DCLK2 inhibitor plus a GM-CSF inhibitor plus a VEGF inhibitor.

In certain embodiments, the treatment may comprise but is not limited to administration of a DCLK1 inhibitor plus a GM-CSF inhibitor plus an IL-6 inhibitor; a DCLK2 inhibitor plus a GM-CSF inhibitor plus an IL-6 inhibitor; and a DCLK1 inhibitor plus a DCLK2 inhibitor plus a GM-CSF inhibitor plus an IL-6 inhibitor.

In certain embodiments, the treatment may comprise but is not limited to administration of an anti-DCLK1 antibody; an anti-DCLK2 antibody; an anti-DCLK1 antibody plus an anti-DCLK2 antibody; an anti-DCLK1 antibody plus an anti-S100A9 antibody; an anti-DCLK2 antibody plus an anti-S100A9 antibody; an anti-DCLK1 antibody plus an anti-DCLK2 antibody plus an anti-S100A9 antibody; an anti-DCLK1 antibody plus an anti-calprotectin antibody; an anti-DCLK2 antibody plus an anti-calprotectin antibody; an anti-DCLK1 antibody plus an anti-DCLK2 antibody plus an anti-calprotectin antibody; an anti-DCLK1 antibody plus an anti-S100A4 antibody; an anti-DCLK2 antibody plus an anti-S100A4 antibody; an anti-DCLK1 antibody plus an anti-DCLK2 antibody plus an anti-S100A4 antibody; an anti-DCLK1 antibody plus an anti-GM-CSF antibody; an anti-DCLK2 antibody plus an anti-GM-CSF antibody; an anti-DCLK1 antibody plus an anti-DCLK2 antibody plus an anti-GM-CSF antibody; an anti-DCLK1 antibody plus an anti-VEGF antibody; an anti-DCLK2 antibody plus an anti-VEGF antibody; an anti-DCLK1 antibody plus an anti-DCLK2 antibody plus an anti-VEGF antibody; an anti-DCLK1 antibody plus an anti-IL-6 antibody; an anti-DCLK2 antibody plus an anti-IL-6 antibody; and an anti-DCLK1 antibody plus an anti-DCLK2 antibody plus an anti-IL-6 antibody.

In certain embodiments, the treatment may comprise but is not limited to administration of an anti-DCLK1 antibody plus an anti-S100A9 antibody plus an anti-calprotectin antibody; an anti-DCLK2 antibody plus an anti-S100A9 antibody plus an anti-calprotectin antibody; and an anti-DCLK1 antibody plus an anti-DCLK2 antibody plus an anti-S100A9 antibody plus an anti-calprotectin antibody.

In certain embodiments, the treatment may comprise but is not limited to administration of an anti-DCLK1 antibody plus an anti-S100A9 antibody plus an anti-S100A4 antibody; an anti-DCLK2 antibody plus an anti-S100A9 antibody plus an anti-S100A4 antibody; and an anti-DCLK1 antibody plus an anti-DCLK2 antibody plus an anti-S100A9 antibody plus an anti-S100A4 antibody.

In certain embodiments, the treatment may comprise but is not limited to administration of an anti-DCLK1 antibody plus an anti-S100A9 antibody plus an anti-GM-CSF antibody; an anti-DCLK2 antibody plus an anti-S100A9 antibody plus an anti-GM-CSF antibody; and an anti-DCLK1 antibody plus an anti-DCLK2 antibody plus an anti-S100A9 antibody plus an anti-GM-CSF antibody.

In certain embodiments, the treatment may comprise but is not limited to administration of an anti-DCLK1 antibody plus an anti-S100A9 antibody plus an anti-VEGF antibody; an anti-DCLK2 antibody plus an anti-S100A9 antibody plus an anti-VEGF antibody; and an anti-DCLK1 antibody plus an anti-DCLK2 antibody plus an anti-S100A9 antibody plus an anti-VEGF antibody.

In certain embodiments, the treatment may comprise but is not limited to administration of an anti-DCLK1 antibody plus an anti-S100A9 antibody plus an anti-IL-6 antibody; an anti-DCLK2 antibody plus an anti-S100A9 antibody plus an anti-IL-6 antibody; and an anti-DCLK1 antibody plus an anti-DCLK2 antibody plus an anti-S100A9 antibody plus an anti-IL-6 antibody.

In certain embodiments, the treatment may comprise but is not limited to administration of a small molecule DCLK1 inhibitor; a small molecule DCLK2 inhibitor; a small molecule DCLK1 inhibitor plus a small molecule DCLK2 inhibitor; a small molecule DCLK1 inhibitor plus a small molecule S100A9 inhibitor; a small molecule DCLK2 inhibitor plus a small molecule S100A9 inhibitor; a small molecule DCLK1 inhibitor plus a small molecule DCLK2 inhibitor plus a small molecule S100A9 inhibitor; a small molecule DCLK1 inhibitor plus a small molecule calprotectin inhibitor; a small molecule DCLK2 inhibitor plus a small molecule calprotectin inhibitor; a small molecule DCLK1 inhibitor plus a small molecule DCLK2 inhibitor plus a small molecule calprotectin inhibitor; a small molecule DCLK1 inhibitor plus a small molecule S100A4 inhibitor; a small molecule DCLK2 inhibitor plus a small molecule S100A4 inhibitor; a small molecule DCLK1 inhibitor plus a small molecule DCLK2 inhibitor plus a small molecule S100A4 inhibitor; a small molecule DCLK1 inhibitor plus a small molecule GM-CSF inhibitor; a small molecule DCLK2 inhibitor plus a small molecule GM-CSF inhibitor; a small molecule DCLK1 inhibitor plus a small molecule DCLK2 inhibitor plus a small molecule GM-CSF inhibitor; a small molecule DCLK1 inhibitor plus a small molecule VEGF inhibitor; a small molecule DCLK2 inhibitor plus a small molecule VEGF inhibitor; a small molecule DCLK1 inhibitor plus a small molecule DCLK2 inhibitor plus a small molecule VEGF inhibitor; a small molecule DCLK1 inhibitor plus a small molecule IL-6 inhibitor; a small molecule DCLK2 inhibitor plus a small molecule IL-6 inhibitor; and a small molecule DCLK1 inhibitor plus a small molecule DCLK2 inhibitor plus a small molecule IL-6 inhibitor.

In certain embodiments, the treatment may comprise but is not limited to administration of a small molecule DCLK1 inhibitor plus a small molecule S100A9 inhibitor plus a small molecule calprotectin inhibitor; a small molecule DCLK2 inhibitor plus a small molecule S100A9 inhibitor plus a small molecule calprotectin inhibitor; and a small molecule DCLK1 inhibitor plus a small molecule DCLK2 inhibitor plus a small molecule S100A9 inhibitor plus a small molecule calprotectin inhibitor.

In certain embodiments, the treatment may comprise but is not limited to administration of a small molecule DCLK1 inhibitor plus a small molecule S100A9 inhibitor plus a small molecule S100A4 inhibitor; a small molecule DCLK2 inhibitor plus a small molecule S100A9 inhibitor plus a small molecule S100A4 inhibitor; and a small molecule DCLK1 inhibitor plus a small molecule DCLK2 inhibitor plus a small molecule S100A9 inhibitor plus a small molecule S100A4 inhibitor.

In certain embodiments, the treatment may comprise but is not limited to administration of a small molecule DCLK1 inhibitor plus a small molecule S100A9 inhibitor plus a small molecule GM-CSF inhibitor; a small molecule DCLK2 inhibitor plus a small molecule S100A9 inhibitor plus a small molecule GM-CSF inhibitor; and a small molecule DCLK1 inhibitor plus a small molecule DCLK2 inhibitor plus a small molecule S100A9 inhibitor plus a small molecule GM-CSF inhibitor.

In certain embodiments, the treatment may comprise but is not limited to administration of a small molecule DCLK1 inhibitor plus a small molecule S100A9 inhibitor plus a small molecule VEGF inhibitor; a small molecule DCLK2 inhibitor plus a small molecule S100A9 inhibitor plus a small molecule VEGF inhibitor; and a small molecule DCLK1 inhibitor plus a small molecule DCLK2 inhibitor plus a small molecule S100A9 inhibitor plus a small molecule VEGF inhibitor.

In certain embodiments, the treatment may comprise but is not limited to administration of a small molecule DCLK1 inhibitor plus a small molecule S100A9 inhibitor plus a small molecule IL-6 inhibitor; a small molecule DCLK2 inhibitor plus a small molecule S100A9 inhibitor plus a small molecule IL-6 inhibitor; and a small molecule DCLK1 inhibitor plus a small molecule DCLK2 inhibitor plus a small molecule S100A9 inhibitor plus a small molecule IL-6 inhibitor.

In certain embodiments, the treatment may comprise but is not limited to administration of an anti-DCLK1 RNA; an anti-DCLK2 RNA; an anti-DCLK1 RNA plus an anti-DCLK2 RNA; an anti-DCLK1 RNA plus an anti-S100A9 RNA; an anti-DCLK2 RNA plus an anti-S100A9 RNA; an anti-DCLK1 RNA plus an anti-DCLK2 RNA plus an anti-S100A9 RNA; an anti-DCLK1 RNA plus an anti-calprotectin RNA; an anti-DCLK2 RNA plus an anti-calprotectin RNA; an anti-DCLK1 RNA plus an anti-DCLK2 RNA plus an anti-calprotectin RNA; an anti-DCLK1 RNA plus an anti-S100A4 RNA; an anti-DCLK2 RNA plus an anti-S100A4 RNA; an anti-DCLK1 RNA plus an anti-DCLK2 RNA plus an anti-S100A4 RNA; an anti-DCLK1 RNA plus an anti-GM-CSF RNA; an anti-DCLK2 RNA plus an anti-GM-CSF RNA; an anti-DCLK1 RNA plus an anti-DCLK2 RNA plus an anti-GM-CSF RNA; an anti-DCLK1 RNA plus an anti-VEGF RNA; an anti-DCLK2 RNA plus an anti-VEGF RNA; an anti-DCLK1 RNA plus an anti-DCLK2 RNA plus an anti-VEGF RNA; an anti-DCLK1 RNA plus an anti-IL-6 RNA; an anti-DCLK2 RNA plus an anti-IL-6 RNA; and an anti-DCLK1 RNA plus an anti-DCLK2 RNA plus an anti-IL-6 RNA.

Inhibitors of DCLK1 and DCLK2, and isoforms thereof, which may be used herein include, but are not limited to:
1. Small molecule inhibitors such as but not limited to DCLK1-IN-1 [ref. 1], Z-TMS [ref. 2], LRRK2-IN-1 [ref. 3], and XMD8-92 [ref. 4], and salts and derivatives or congeners thereof;
2. Polyclonal and monoclonal antibodies and antibody fragments having specific binding for DCLK1 and/or DCLK2, and isoforms thereof [refs. 12, 17, 18];
3. siRNAs and shRNAs [refs. 5-7, 12] that knock down expression of DCLK1 and/or DCLK2;
4. CRISPR-CAS-mediated knockdown of DCLK1 and/or DCLK2 genes; and
5. Inhibitors of DCLK1 and/or DCLK2 promoters.

Inhibitors of S100A9 and calprotectin (S100A8/S100A9 complex) or its nearest family members with similar activities (e.g., S100A4), which may be used herein include, but are not limited to:
1. Small molecule inhibitors such as but not limited to: Tasquinimod (TasQ) [ref. 8] and Paquinimod and their analogues or derivatives, bromodomain inhibitor JQ1, certain N-(2-oxo-3-(trifluoromethyl)-2,3-dihydro-1H-benzo[d]imidazo[1,2-a]imidazo-l-3-yl)amide derivatives [ref. 15], and certain N-(heteroaryl)-sulfonamide derivatives [ref. 16];
2. Antibodies and fragments thereof [ref. 14] which block S100A9 activity, expression and/or interaction with its binding partners (e.g., receptors and S100A8); and
3. Nucleic acid based downregulators of S100A9 levels.

Inhibitors of other DCLK-associated agents, including but not limited to:
1. Inhibitors of other kinases and therapeutic targets such as immune ligands and immune receptors;
2. Inhibitors of cytokines and growth factor inhibitors (e.g., inhibitors of GM-CSF [ref. 9] such as lenzilumab, mavrilimumab, or otilimab and anti-VEGF inhibitors such as bevacizumab [ref. 10]);
3. Inhibitors of IL-6 (sarilumab, tociluzumab) [ref. 11]; and
4. Antibodies and immunotherapeutic agents.

As noted, in non-limiting embodiments, the DCLK1, DCLK2, S100A9, calprotectin, S100A4, GM-CSF, VEGF, and IL inhibitors may be anti-DCLK1 antibodies, anti-DCLK2 antibodies, anti-S100A9 antibodies, anti-calprotectin antibodies, anti-S100A4 antibodies, anti-GM-CSF antibodies, anti-VEGF antibodies, and anti-IL antibodies, respectively. The term “antibody” as used herein can refer to both intact, “full length” antibodies as well as to portions or fragments of the anti-DCLK1 antibodies, anti-DCLK2 antibodies, anti-S100A9 antibodies, anti-calprotectin antibodies, anti-S100A4 antibodies, anti-GM-CSF antibodies, anti-VEGF antibodies, and anti-IL antibodies which are able to bind to DCLK1, DCLK2, S100A9, calprotectin, S100A4, GM-CSF, VEGF, and IL, respectively (also referred to herein as antigen binding fragments, antigen binding portions, binding fragments, or binding portions) thereof. As used herein, the term “antibody” includes, but is not limited to, synthetic antibodies, monoclonal antibodies, recombinantly produced antibodies, intrabodies, multispecific antibodies (including bi-specific antibodies), human antibodies, humanized antibodies, chimeric antibodies, recombinant single chain polypeptide molecules in which light and heavy chain variable regions are connected by a peptide linker, i.e., single-chain Fv (scFv) fragments, bivalent scFv (bi-scFv), trivalent scFv (tri-scFv), Fab fragments, Fab′ fragments, F(ab′) fragments, F(ab′)₂ fragments, F(ab)₂ fragments, disulfide-linked Fvs (sdFv) (including bi-specific sdFvs), and anti-idiotypic (anti-Id) antibodies, diabodies, dAb fragments, nanobodies, diabodies, triabodies, tetrabodies, linear antibodies, isolated CDRs, and epitope-binding fragments of any of the above. Regardless of structure, an antibody fragment binds with the same antigen that is recognized by the intact antibody. For example, an anti-DCLK1 antibody fragment binds with an epitope of DCLK1, an anti-DCLK2 antibody fragment binds with an epitope of DCLK2, and an anti-S100A9 antibody fragment binds with an epitope of S100A9. Fragments can be produced by recombinant DNA techniques or by enzymatic or chemical separation of intact immunoglobulins.

The antibodies of several embodiments provided herein may be monospecific, bispecific, trispecific, or of greater multispecificity, such as multispecific antibodies formed from antibody fragments. The term “antibody” also includes a diabody (homodimeric Fv fragment) or a minibody (V_{L}-V-C_{H3}), a bispecific antibody, or the like. A bispecific or bifunctional antibody is an artificial hybrid antibody having two different heavy/light chain pairs and two different binding sites. For example a bispecific antibody may be constructed to be able to bind to both DCLK1 and DCLK2, for example, or to both DCLK1 and S100A9, for example, or to both DCLK2 and S100A9. A trispecific antibody may be able to bind to all three of DCLK1, DCLK2, and S100A9, for example. Multispecific antibodies may be specific for different epitopes of a polypeptide or may be specific for both a polypeptide as well as for a heterologous epitope, such as a heterologous polypeptide or solid support material. Single chain antibodies produced by joining antibody fragments using recombinant methods, or a synthetic linker, are also encompassed by the present disclosure (e.g., see, for example, International Patent Application Publication Nos. WO 93/17715; WO 92/08802; WO 91/00360; and WO 92/05793; and U.S. Pat. Nos. 4,474,893; 4,714,681; 4,925,648; 5,573,920; and 5,601,819).

The term “monoclonal antibody” as used herein refers to an antibody obtained from a population of substantially homogeneous antibodies, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method. For example, the monoclonal antibodies to used in accordance with the present disclosure can be made by the hybridoma method first described by Kohler et al. (Nature, 256:495 (1975)), or may be made by recombinant DNA methods (see, for example, U.S. Pat. No. 4,816,567).

The compositions, formulations, and methods described herein may include monoclonal antibodies. Rodent monoclonal antibodies to specific antigens may be obtained by methods known to those skilled in the art (e.g., see Kohler and Milstein, op.cit., and Coligan et al. (eds.), Current Protocols in Immunology, Vol. 1, pages 2.5.1-2.6.7 (John Wiley & Sons 1991)). General techniques for cloning murine immunoglobulin variable domains have been disclosed, for example, by the publication of Orlandi et al. (Proc. Nat'l Acad. Sci. USA, 86: 3833 (1989)).

An “isolated” antibody refers to an antibody that has been identified and separated and/or recovered from components of its natural environment and/or an antibody that is recombinantly produced. A “purified antibody” is an antibody that is typically at least 50% w/w pure of interfering proteins and other contaminants arising from its production or purification but does not exclude the possibility that the monoclonal antibody is combined with an excess of pharmaceutical acceptable carrier(s) or other vehicle(s) intended to facilitate its use. Interfering proteins and other contaminants can include, for example, cellular components of the cells from which an antibody is isolated or recombinantly produced. Sometimes monoclonal antibodies are at least 60%, at least 70%, at least 80%, at least 90%, at least 95%, or at least 99% w/w pure of interfering proteins and contaminants from production or purification. The antibodies described herein, including murine, chimeric, and humanized antibodies, can be provided in isolated and/or purified form.

A “therapeutic agent” is an atom, molecule, radiation, or compound that is useful in the treatment of a disease. Examples of therapeutic agents include but are not limited to antibodies, antibody fragments, drugs, cytokine or chemokine inhibitors, pro-apoptotic agents, tyrosine kinase inhibitors, toxins, enzymes, nucleases, hormones, immunomodulators, antisense oligonucleotides, siRNA, RNAi, chelators, boron compounds, photoactive agents, dyes, radiation, and radioisotopes.

A “diagnostic agent” is an atom, molecule, or compound that is useful in diagnosing a disease. Useful diagnostic agents include, but are not limited to, radiation, radioisotopes, dyes, contrast agents, fluorescent compounds or molecules, and enhancing agents (e.g., paramagnetic ions). In certain particular (but non-limiting) embodiments, the diagnostic agents are selected from the group comprising radioisotopes, enhancing agents, and fluorescent compounds.

An “immunoconjugate” or “antibody-drug conjugate” is a conjugate of an antibody with an atom, molecule, or a higher-ordered structure (e.g., with a liposome), a therapeutic agent, or a diagnostic agent. The term “antibody” as used herein can also refer to both intact antibodies, and to DCLK1-binding fragments, which are conjugated to a therapeutic agent (e.g., a cytotoxic or cytostatic drug) or to a diagnostic agent.

As used herein, the term “antibody fusion protein” is a recombinantly produced antigen-binding molecule in which an antibody or antibody fragment is linked to another protein or peptide, such as the same or different antibody or antibody fragment. The fusion protein may comprise a single antibody component, a multivalent or multispecific combination of different antibody components, or multiple copies of the same antibody component. The fusion protein may additionally comprise an antibody or an antibody fragment and a therapeutic agent.

For purposes of classifying amino acids substitutions as conservative or nonconservative, amino acids are grouped in one non-limiting embodiment as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same group. Non-conservative substitutions constitute exchanging a member of one of these groups for a member of another.

Tables of conservative amino acid substitutions have been constructed and are known in the art. In other embodiments, examples of interchangeable amino acids include, but are not limited to, the following: arginine and lysine; glutamate and aspartate; serine and threonine; glutamine and asparagine; and valine, leucine, and isoleucine. In other non-limiting embodiments, the following substitutions can be made: Ala (A) by leu, ile, or val; Arg (R) by gln, asn, or lys; Asn (N) by his, asp, lys, arg, or gln; Asp (D) by asn or glu; Cys (C) by ala or ser; Gln (Q) by glu or asn; Glu (E) by gln or asp; Gly (G) by ala; His (H) by asn, gln, lys, or arg; Ile (I) by val, met, ala, phe, or leu; Leu (L) by val, met, ala, phe, or ile; Lys (K) by gln, asn, or arg; Met (M) by phe, ile, or leu; Phe (F) by leu, val, ile, ala, or tyr; Pro (P) by ala; Ser (S) by thr; Thr (T) by ser; Trp (W) by phe or tyr; Tyr (Y) by trp, phe, thr, or ser; and Val (V) by ile, leu, met, phe, or ala.

Other considerations for amino acid substitutions include whether or not the residue is located in the interior of a protein or is solvent—(i.e., externally) exposed. For interior residues, conservative substitutions include for example: Asp and Asn; Ser and Thr; Ser and Ala; Thr and Ala; Ala and Gly; Ile and Val; Val and Leu; Leu and Ile; Leu and Met; Phe and Tyr; and Tyr and Trp. For solvent-exposed residues, conservative substitutions include for example: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; and Phe and Tyr.

Percentage sequence identities can be determined with antibody sequences maximally aligned by the Kabat numbering convention. After alignment, if a particular antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises an antibody may contain the antibody alone or in combination with other ingredients.

The phrase “pharmaceutically acceptable salt” refers to pharmaceutically acceptable organic or inorganic salts of a DCLK1, DCLK2, or S100A9 inhibitor. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as (but not limited to) an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterions.

A humanized antibody is a genetically engineered antibody in which the variable heavy and variable light CDRs from a non-human “donor” antibody are grafted into human “acceptor” antibody sequences (see for example, U.S. Pat. Nos. 5,530,101; 5,585,089; 5,225,539; 6,407,213; 5,859,205; and 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a non-human donor antibody and variable region framework sequences and constant regions, if present, entirely or substantially from human antibody sequences. Similarly, a humanized heavy chain has at least one, two, and usually all three CDRs entirely or substantially from a donor antibody heavy chain, and a heavy chain variable region framework sequence, and heavy chain constant region, if present, substantially from human heavy chain variable region framework and constant region sequences. Similarly, a humanized light chain has at least one, two, and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain.

The inhibitors used in the present compositions and methods of treatment can be formulated into compositions for delivery to a mammalian subject. The composition can be administered alone and/or mixed with a pharmaceutically acceptable vehicle or excipient. Suitable vehicles are, for example (but not by way of limitation), water, saline, dextrose, glycerol, ethanol, or the like, and combinations thereof. In addition, the vehicle can contain minor amounts of auxiliary substances such as (but not limited to) wetting or emulsifying agents, pH buffering agents, or adjuvants. The compositions of the present disclosure can also include ancillary substances, such as (but not limited to) pharmacological agents, cytokines, or other biological response modifiers.

Furthermore, the compositions can be formulated into compositions in either neutral or salt forms. Pharmaceutically acceptable salts include (but are not limited to) the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, and procaine.

Compositions can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The compositions can be combined with a pharmaceutically acceptable carrier (excipient) to form a pharmacological composition. Pharmaceutically acceptable carriers can contain a physiologically acceptable compound that acts to, for example but not by way of limitation) stabilize or increase or decrease the absorption or clearance rates of the pharmaceutical compositions. Physiologically acceptable compounds can include, for example but not by way of limitation: carbohydrates, such as glucose, sucrose, or dextrans; antioxidants, such as ascorbic acid or glutathione; chelating agents; low molecular weight proteins; detergents; liposomal carriers; excipients; or other stabilizers and/or buffers. Other physiologically acceptable compounds include (but are not limited to) wetting agents, emulsifying agents, dispersing agents, or preservatives.

When administered orally, the present compositions may be protected from digestion. This can be accomplished either by complexing the inhibitor thereof with a composition to render it resistant to acidic and enzymatic hydrolysis or by packaging the inhibitor in an appropriately resistant carrier such as (but not limited to) a liposome, e.g., such as shown in U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The present compositions can also be administered in sustained delivery or sustained release mechanisms. For example, biodegradeable microspheres or capsules or other biodegradeable polymer configurations capable of sustained delivery of a peptide can be included herein.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include (for example but not by way of limitation) air jet nebulizers.

The inhibitor can be delivered alone or as pharmaceutical compositions by any means known in the art, such as (but not limited to) systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

In one aspect, the pharmaceutical formulations are incorporated in lipid monolayers or bilayers, such as (but not limited to) liposomes, such as shown in U.S. Pat. Nos. 6,110,490; 6,096,716; 5,283,185; and 5,279,833. In other aspects, non-limiting embodiments of the disclosure include formulations in which the polypeptides or nucleic acids have been attached to the surface of the monolayer or bilayer of the liposomes. Liposomes and liposomal formulations can be prepared according to standard methods and are also well known in the art, such as (but not limited to) those disclosed in U.S. Pat. Nos. 4,235,871; 4,501,728; and 4,837,028.

In one aspect, the compositions are prepared with carriers that will protect the inhibitor against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The subject inhibitors in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active compound is combined in a mixture with one or more pharmaceutically suitable excipients. Sterile phosphate-buffered saline is one non-limiting example of a pharmaceutically suitable excipient.

Non-limiting examples of routes of administration of the compositions described herein include parenteral injection, e.g., by subcutaneous, intramuscular, or transdermal delivery. Other forms of parenteral administration include (but are not limited to) intravenous, intraarterial, intralymphatic, intrathecal, intraocular, intracerebral, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives.

Formulated compositions comprising the inhibitors can be used (for example but not by way of limitation) for subcutaneous, intramuscular, or transdermal administration. Compositions can be presented in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents. Alternatively, the compositions can be in powder form for constitution with a suitable vehicle, e.g., sterile pyrogen-free water, before use.

The compositions may be administered in solution. The formulation thereof may be in a solution having a suitable pharmaceutically acceptable buffer, such as (but not limited to) phosphate, Tris (hydroxymethyl) aminomethane-HCl, or citrate, and the like. Buffer concentrations should be in the range of 1 to 100 mM. The formulated solution may also contain a salt, such as (but not limited to) sodium chloride or potassium chloride in a concentration of 50 to 150 mM. An effective amount of a stabilizing agent such as (but not limited to) mannitol, trehalose, sorbitol, glycerol, albumin, a globulin, a detergent, a gelatin, a protamine, or a salt of protamine may also be included.

Exemplary, non-limiting ranges for a therapeutically or prophylactically effective amount of an inhibitor, such as (but not limited to) for a DCLK1, DCLK2, or S100A9 inhibitor, include a range of from about 0.001 mg/kg of the subject's body weight to about 100 mg/kg of the subject's body weight, such as but not limited to a range of from about 0.01 mg/kg to about 50 mg/kg, a range of from about 0.1 mg/kg to about 50 mg/kg, a range of from about 0.1 mg/kg to about 40 mg/kg, a range of from about 1 mg/kg to about 30 mg/kg, a range of from about 1 mg/kg to about 20 mg/kg, a range of from about 2 mg/kg to about 30 mg/kg, a range of from about 2 mg/kg to about 20 mg/kg, a range of from about 2 mg/kg to about 15 mg/kg, a range of from about 2 mg/kg to about 12 mg/kg, a range of from about 2 mg/kg to about 10 mg/kg, a range of from about 3 mg/kg to about 30 mg/kg, a range of from about 3 mg/kg to about 20 mg/kg, a range of from about 3 mg/kg to about 15 mg/kg, a range of from about 3 mg/kg to about 12 mg/kg, or a range of from about 3 mg/kg to about 10 mg/kg, or a range of from about 10 mg to about 1500 mg as a fixed dosage.

The composition is formulated to contain an effective amount of the inhibitor, wherein the amount depends on the subject to be treated and the severity of the condition of the subject. In certain non-limiting embodiments, the present inhibitor may be administered at a dose ranging from about 0.001 mg to about 10 g, from about 0.01 mg to about 10 g, from about 0.1 mg to about 10 g, from about 1 mg to about 10 g, from about 1 mg to about 9 g, from about 1 mg to about 8 g, from about 1 mg to about 7 g, from about 1 mg to about 6 g, from about 1 mg to about 5 g, from about 10 mg to about 10 g, from about 50 mg to about 5 g, from about 50 mg to about 5 g, from about 50 mg to about 2 g, from about 0.05 μg to about 1.5 mg, from about 10 μg to about 1 mg protein, from about 30 μg to about 500 μg, from about 40 μg to about 300 μg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, or from about 1 mg to about 2 mg. The specific dose level for any particular subject depends upon a variety of factors, including (but not limited to) the activity of the specific inhibitor, the age, body weight, general health, sex, diet, time of administration, route of administration, and rate of excretion, the drug combination, and the severity of the disease in the subject undergoing therapy.

The dosage of an administered inhibitor for humans will vary depending upon factors such as (but not limited to) the patient's age, weight, height, sex, general medical condition, and previous medical history. In certain non-limiting embodiments, the recipient is provided with a dosage of the inhibitor(s) that is in the range of from about 1 mg to about 1000 mg as a single infusion or single or multiple injections, although a lower or higher dosage also may be administered. In certain non-limiting embodiments, the dosage may be in the range of from about 25 mg to about 100 mg per square meter (m²) of body surface area for a typical adult, although a lower or higher dosage also may be administered. Non-limiting examples of dosages that may be administered to a human subject further include 1 to 500 mg, 1 to 70 mg, or 1 to 20 mg, although higher or lower doses may be used. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In some non-limiting embodiments, the amount of a DCLK1, DCLK2, and/or S100A9 inhibitor effective as a treatment against COVID-19 is in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 75 μM, about 80 μM, about 90 μM, about 100 μM, about 125 μM, about 150 μM, about 175 μM, about 200 μM, about 250 μM, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

In some non-limiting methods, the patient is administered the inhibitor every one, two, three, or four weeks, for example. The dosage depends on the frequency of administration, condition of the patient, response to prior treatment (if any), whether the treatment is prophylactic or therapeutic, and whether the disorder is acute or chronic, among other factors.

Administration can be (for example but not by way of limitation) parenteral, intravenous, oral, subcutaneous, intra-arterial, intracranial, intrathecal, intraperitoneal, topical, intranasal, or intramuscular. Administration can also be localized directly into a tumor. Administration into the systemic circulation by intravenous or subcutaneous administration is typical. Intravenous administration can be, for example (but not by way of limitation), by infusion over a period such as (but not limited to) 30-90 min or by a single bolus injection.

The number of dosages administered depends on the severity of the condition and the response to therapy (e.g., whether presenting acute or chronic symptoms) Treatment can be repeated for recurrence of an acute disorder or acute exacerbation. For chronic disorders, the inhibitor can be administered at regular intervals, such as (but not limited to) weekly, fortnightly, monthly, quarterly, every six months for at least 1, 5, or 10 years, or for the life of the patient if the condition is chronic.

In certain non-limiting embodiments, pharmaceutical compositions for parenteral administration are sterile, substantially isotonic, and manufactured under GMP conditions. Pharmaceutical compositions can be provided in unit dosage form (i.e., the dosage for a single administration). Pharmaceutical compositions can be formulated using one or more physiologically acceptable carriers, diluents, excipients, or auxiliaries. The formulation depends on the route of administration chosen. For injection, inhibitors can be formulated in aqueous solutions, such as (but not limited to) in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively the inhibitors can be in lyophilized form for constitution with a suitable vehicle, e.g., sterile pyrogen-free water, before use.

Some non-limiting embodiments provided herein include kits. In some non-limiting embodiments, a kit can include any of the inhibitors as described or otherwise contemplated herein, In some non-limiting embodiments, the inhibitor is lyophilized. In some non-limiting embodiments, the inhibitor is in aqueous solution, or other carrier as described herein. In some non-limiting embodiments, the kit includes a pharmaceutical carrier for administration of the inhibitor. Certain non-limiting embodiments of the present disclosure include kits containing components suitable for treatments or diagnosis. Exemplary kits may contain at least one inhibitor as described herein. A device capable of delivering the kit components by injection, for example, a syringe for subcutaneous injection, may be included in some non-limiting embodiments. Where transdermal administration is used, a delivery device such as hollow microneedle delivery device may be included in the kit in some non-limiting embodiments. Exemplary transdermal delivery devices are known in the art, such as (but not limited to) a hollow Microstructured Transdermal System (e.g., 3M Corp.), and any such known device may be used. The kit components may be packaged together or separated into two or more containers. In some non-limiting embodiments, the containers may be vials that contain sterile, lyophilized formulations of a composition that are suitable for reconstitution. A kit may also contain one or more buffers suitable for reconstitution and/or dilution of other reagents. Alternatively, the inhibitor may be delivered and stored as a liquid formulation. Other containers that may be used include, but are not limited to, a pouch, tray, box, tube, or the like. Kit components may be packaged and maintained sterilely within the containers. Another component that can be included is instructions for the use of the kit for treatment.

Severe acute respiratory distress syndrome (ARDS), hypoxia, and cytokine storm commonly observed in COVID-19 patients with comorbidities are contributed by the SARS-CoV-2-induced host factors. Disclosed herein is evidence that SARS-CoV-2 triggers abundant co-expression of DCLK1, a tumor stem cell regulator, and of S100A9, a pro-inflammatory protein in lung, liver, and peripheral blood mononuclear immune cells. Induction of these proteins markedly upregulates SARS-CoV-2 production and generates a cytokine storm signature (TNFα, IL-1β, IL-6, IL-10, M-CSF, GM-CSF) analogous to that observed in severe COVID-19. A DCLK1 kinase-specific inhibitor (DCLK1-IN-1) simultaneously diminished the cytokine storm signature and virus production. Additionally, a decrease in DCLK1 expression by the co-treatment with DCLK1-IN-1 with and S100A9 inhibitor (tasquinimod) was accompanied by inhibition of active caspase 1 and normalization of the interferon response, demonstrating the clinical significance of this therapeutic approach. Circulating DCLK1⁺S100A9⁺ mononuclear immune cells correlated with the severity of COVID-19. The alveoli and hepatic sinusoidal DCLK1⁺S100A9⁺ cells were identified as CD206⁺ M2 macrophage and hypersegmented neutrophils. The infected lung cells also induced CD68⁺/CD86⁺ polarized pro-inflammatory M1 macrophages in peripheral blood mononuclear cells. The evidence demonstrates that DCLK1 plays a critical role in SARS-CoV-2 viremia, hyperinflammation, and immune evasion by dysregulating tissue-resident macrophages and peripheral mononuclear cells. The results demonstrate that DCLK1 and S100A9 can be used as therapeutic targets to treat patients with severe COVID-19, particularly those who have comorbid factors which upregulate DCLK1.

DCLK1 is a multifunctional kinase best known for its involvement in clonogenicity, stemness, and tumorigenesis. It synthesizes microtubules that facilitate molecular transport and contains a kinase domain that phosphorylates multiple substrates including the Spen family transcriptional repressor (SPEN) and cyclin dependent kinase 11B (CDK11B). It had been previously shown that DCLK1 is induced by tissue injury and inflammation but is not otherwise expressed to any degree in liver, lung, or gastrointestinal tract. Other previous work showed that DCLK1 enhances replication of hepatitis C virus (HCV), a positive-strand RNA virus. In contrast, downregulation of DCLK1 inhibits viral replication and growth of hepatocellular carcinoma in xenograft models. However, prior to the present work, the role or importance of DCLK1 during SARS-CoV-2 infection had not been established. Herein, we have identified DCLK1 as a key mediator for SARS-CoV-2 production in lung and liver cells and show that that DCLK1 facilitates the development of the cytokine storm and viral immune evasion in COVID-19 via dysregulation of tissue-resident macrophages and peripheral blood mononuclear cells.

DCLK1 also upregulates S100A9. During inflammation, S100A9 is expressed by many epithelial and immune cells (e.g., hepatocytes, monocytes, macrophages, and neutrophils). This calcium-binding protein forms homo- and heteromers with S100A8 that activate NF-κB via interactions with Toll-like receptor 4 (TLR4) and receptors for advanced glycation end products (RAGE). This leads to the expression of IL-1β, IL-6, TNF-α, IL-10, and inducible nitric oxide synthase (iNOS). Reactive oxygen species (ROS) are also generated by monocytes, macrophages, and neutrophils. DCLK1 and S100A9 expression is regulated via NF-κB and a feed-forward mechanism. In the present disclosure we also show that DCLK1 and S100A9 inhibitors can reduce viral replication and limit hyperinflammation seen in COVID-19 patients, particularly in the context of comorbid chronic liver disease.

In one embodiment, the present disclosure is directed to a method of treating severe or critical COVID-19 in a subject in need of such therapy, comprising administering to the subject an inhibitor of at least one of DCLK1 and DCLK2, and optionally an inhibitor of S100A9 or GM-CSF.

In one embodiment, the present disclosure is directed to a composition comprising at least one of DCLK1 and DCLK2, and optionally an inhibitor of S100A9 or GM-CSF, for use in the treatment of severe or critical COVID-19 in a subject in need of such therapy.

In another embodiment, the present disclosure is directed to a method of inhibiting SARS-CoV-2 in a host, comprising administering to the host an inhibitor of at least one DCLK1 and DCLK2, and optionally an inhibitor of S100A9 or GM-CSF.

In another embodiment, the present disclosure is directed to a composition comprising at least one of DCLK1 and DCLK2, and optionally an inhibitor of S100A9 or GM-CSF, for use in inhibiting SARS-CoV-2 in a host.

In another embodiment, the present disclosure is directed to a method of treating COVID-19-associated cytokine storm in a subject in need of such therapy, comprising administering to the subject an inhibitor of at least one of DCLK1 and DCLK2, and optionally an inhibitor of S100A9 or GM-CSF.

In another embodiment, the present disclosure is directed to a composition comprising at least one of DCLK1 and DCLK2, and optionally an inhibitor of S100A9 or GM-CSF, for use in the treatment of COVID-19-associated cytokine storm in a subject in need of such therapy.

In another embodiment, the present disclosure is directed to a method of determining if a patient having coronavirus infectious disease-19 should be administered a treatment protocol for severe or critical COVID-19, comprising (1) obtaining a sample of mononuclear cells from the patient and quantifying the number of DCLK1+S100A9+ mononuclear cells in the cell sample, and (2) administering to the patient the treatment protocol for severe or critical COVID-19 when the number of DCLK1+S100A9+ mononuclear cells in the cell sample exceeds a predetermined threshold for DCLK1+S100A9+ mononuclear cells in the cell sample by at least two-fold. The predetermined threshold for DCLK1+S100A9+ mononuclear cells may be calculated as an average of the number of DCLK1+S100A9+ mononuclear cells measured in a population of normal subjects. The treatment protocol for severe or critical COVID-19 may be administered when the number of DCLK1+S100A9+ mononuclear cells in the cell sample exceeds the predetermined threshold by at least four-fold.

In another embodiment, the present disclosure is directed to a method of determining if a patient having coronavirus infectious disease-19 should be administered a treatment protocol for severe or critical COVID-19, comprising (1) quantifying the number of DCLK1+S100A9+ mononuclear cells in a sample of the patient's mononuclear cells, and (2) selecting the patient for the treatment protocol for severe or critical COVID-19 when the number of DCLK1+S100A9+ mononuclear cells in the sample exceeds a predetermined threshold for DCLK1+S100A9+ mononuclear cells in the sample by at least two-fold. The predetermined threshold for DCLK1+S100A9+ mononuclear cells may be calculated as an average of the number of DCLK1+S100A9+ mononuclear cells measured in a population of normal subjects. The treatment protocol for severe or critical COVID-19 may be administered when the number of DCLK1+S100A9+ mononuclear cells in the cell sample exceeds the predetermined threshold by at least four-fold.

In another embodiment, the present disclosure is directed to a method of determining if a patient having COVID-19 should be administered a treatment protocol for severe or critical COVID-19, comprising (1) obtaining a sample of mononuclear cells from the patient and quantifying the number of DCLK1+S100A9+CD206+ mononuclear cells in the cell sample, and (2) administering to the patient the treatment protocol for severe or critical COVID-19 when the number of DCLK1+S100A9+CD206+ mononuclear cells in the cell sample exceeds a predetermined threshold for DCLK1+S100A9+CD206+ mononuclear cells in the cell sample by at least two-fold. The predetermined threshold for DCLK1+S100A9+CD206+ mononuclear cells may be calculated as an average of the number of DCLK1+S100A9+CD206+ mononuclear cells measured in a population of normal subjects. The treatment protocol for severe or critical COVID-19 may be administered when the number of DCLK1+S100A9+CD206+ mononuclear cells in the cell sample exceeds the predetermined threshold by at least four-fold.

In another embodiment, the present disclosure is directed to a method of determining if a patient having COVID-19 should be administered a treatment protocol for severe or critical COVID-19, comprising (1) quantifying the number of DCLK1+S100A9+CD206+ mononuclear cells in a sample of the patient's mononuclear cells, and (2) selecting the patient for the treatment protocol for severe or critical COVID-19 when the number of DCLK1+S100A9+CD206+ mononuclear cells in the cell sample exceeds a predetermined threshold for DCLK1+S100A9+CD206+ mononuclear cells in the cell sample by at least two-fold. The predetermined threshold for DCLK1+S100A9+CD206+ mononuclear cells may be calculated as an average of the number of DCLK1+S100A9+CD206+ mononuclear cells measured in a population of normal subjects. The treatment protocol for severe or critical COVID-19 may be administered when the number of DCLK1+S100A9+CD206+ mononuclear cells in the cell sample exceeds the predetermined threshold by at least four-fold.

Having generally described embodiments drawn to treatment of COVID-19 in which a subject is administered an inhibitor of DCLK1 and/or DCLK2, and optionally another inhibitor(s) directed to another target(s), a further understanding of the compositions and methods of the disclosure can be obtained by reference to certain specific experiments and examples which are provided below for purposes of illustration only and are not intended to be limiting.

### EXPERIMENTAL

### I.

### Materials and Methods

Collection of Postmortem Specimens, PBMCs, and Sera from COVID-19 Patients.

Forensic pathologists performed complete post-mortem examinations on cases brought to the Oklahoma City Office of the Chief Medical Examiner. The autopsies of individuals (n=11) were performed in accordance with recommended guidelines. The lungs and livers of these cases did not show evidence for autolysis by histological examination and were included in the study. For blood analysis of hospitalized COVID-19 patients (n=18), PBMCs and corresponding sera were obtained from the OSCTR at the OUHSC. Normal blood samples were donated by healthy adults and each tested negative for SARS-CoV-2 (n=3). Normal PBMCs were purchased from ZenBio Inc. Infectious Diseases Society of America guidelines (idsociety.org) were used to classify COVID-19 cases as mild, moderate, and severe/critical. The study was approved by the Institutional Review Board (#12906, dated Feb. 8, 2021).

### Cell Cultures, SARS-CoV-2 Infection, and TCID₅₀ Assay.

Lung adenocarcinoma cells (Calu-3, Cat #HTB-55) were purchased from ATCC. Hepatoma cells (Huh7), and derivative cells expressing recombinant fluorescent protein (Huh7-RFP) and RFP-DCLK1 (Huh7-RFP-DCLK1), have been previously described (Ali N, Chandrakesan P, Nguyen C B, Husain S, Gillaspy A F, Huycke M, Berry W L, May R, Qu D, Weygant N, Sureban S M, Bronze M S, Dhanasekaran D N, Houchen C W. 2015. Inflammatory and oncogenic roles of a tumor stem cell marker doublecortin-like kinase (DCLK1) in virus-induced chronic liver diseases. Oncotarget 6:20327-44; Ali N, Nguyen C B, Chandrakesan P, Wolf R F, Qu D, May R, Goretsky T, Fazili J, Barrett T A, Li M, Huycke M M, Bronze M S, Houchen C W. 2020. Doublecortin-like kinase 1 promotes hepatocyte clonogenicity and oncogenic programming via non-canonical b-catenin-dependent mechanism. Sci Rep 10:10578). Cryopreserved primary human hepatocytes (Corning) and normal PBMCs (ZenBio) were purchased from vendors. All methods were carried out in accordance with the guidelines and regulations of the OUHSC Institutional Biosafety Committee (approval #200470-2440A).

SARS-CoV-2 (USA-WA1/2020 strain) was obtained from BEI Resources at the National Institutes of Allergy and Infectious Diseases in Bethesda, Maryland (Catalog number NR-52281). Coronavirus SARS-CoV-2 infectious clones, icSARS-CoV-2 were obtained from the World Reference Center for Emerging Viruses and Arboviruses through the University of Texas Medical Branch at Galveston, Texas. All experiments related to infectious clones and cultures were performed in a Biosafety Level-3 laboratory after Institutional Biosafety Committee approval (IBC Protocol #100492). SARS-CoV-2 was grown for up to 3 passages in Vero-E6 cells (ATCC: CRL-1586) that were cultured in complete Dulbecco's Modified Eagle's Medium (DMEM) containing 5% FBS and antibiotics (Pen/Strep, Gibco), at 37° C. and 5% CO₂. To passage SARS-CoV-2, Vero-E6 cells were grown in a T-150 flask to 50% confluency (˜10 million cells) and inoculated with SARS-CoV-2 at a multiplicity of infection (MOI) of 0.001. Infection of cells was carried out in 3 mL of DMEM without FBS for 1 h at 37° C. with gentle mixing. Cells were placed in complete media for 48 h. Virus was harvested from spent culture supernatants, centrifuged, and stored at −80° C. All experiments involving viral infection were conducted using viruses from the same stock. SARS-CoV-2 was titrated using the 50% tissue culture infectious dose (TCID₅₀) method (Reed, L. J. 1938. A simple method of estimating fifty percent endpoints. Am J Hyg 27:493-497). Vero-E6 cells were seeded at 10,000 cells per well in a 96-well plate, infected with serially diluted SARS-CoV-2 containing spent media, and cytopathic effects determined after 96 h. The number of virus-positive wells was used to calculate a TCID₅₀/mL at each dilution.

### SARS-CoV-2 Infection of Target Cells in Cocultures and Evaluation of Inhibitors.

Huh7 cells, RFP- or RFP-DCLK1-expressing Huh7 cells, and Calu3 cells were infected for 2 h at 37° C. with SARS-CoV-2 (multiplicity of infection=1). Cell supernatants were collected at 24, 48, 72, and 96 h post-infection to detect productive virus infection. Viral titers in spent media were measured by TCID₅₀. Huh7-RFP-DCLK1 cells were cultured in 6-well plates, infected for 2 h with SARS-COV-2 and DCLK1-IN-1 or DCLK1-NEG added at final concentrations of 0, 0.1, 2.5, 5.0, and 10 μM. The cells were lysed in RIPA buffer (Thermo Fisher), and total lysates were prepared using Bullet Blender's protocol (Next Advance, Inc.). Western blots were carried out with antibodies and fluorescence conjugated reagents and imaging was performed with LI-COR imaging system. For coculture, purified PBMCs (10⁶ per 6-well plate) from normal donors were added to cells 2 h post-infection. The effects of inhibitors were analyzed as above. Band intensities were calculated using Image Studio Digits. Images were produced in compliance with digital image and integrity policies.

### Immunohistochemistry (IHC) and Confocal Microscopy.

De-identified lung and liver tissue slides were subjected to immunofluorescence staining using the Akoya Opal 7-Color IHC kit. Stained slides were imaged and evaluated by confocal microscopy (Leica SP8). The intensity of cellular staining was scored qualitatively by two investigators and consensus achieved. Staining was considered to be absent (−), rare (+/−), weak (+), moderate (++), or strong (+++). H&E-stained slides were used for pathological analyses. Fifty thousand PHHs from normal donors were cultured in a Lab-Teck II chambered system (Thermo Fisher) containing 100 μl Matrigel per well in Hepato-STIM hepatocyte defined medium supplemented with EGF (10 ng/mL). To perform confocal microscopy, cultures were fixed with 10% formamide-PBS for 20 m, blocked with 1% BSA in washing buffer (PBS, 0.5% Triton X-100, 0.05% Tween-20). After washing, the cells were stained with primary and secondary antibodies conjugated with fluorophores. Nuclear staining was carried out with 4′,6-diamidino-2-phenylindole (Dapi). Stained cells were subjected to confocal microscopy after the addition of ProLong Gold Anti-fade reagent (Invitrogen).

### PBMC Preparation, Flow Cytometry, and Cytokine/Chemokine Analysis.

PBMCs from COVID-19 patients were isolated using Lymphocyte Separation Medium (MediaTech/25-072-CV) and resuspended in CS10 solution at 1×10⁶ cells/mL for cryopreservation. Frozen normal PBMCs were stained with Aqua-Zombie to determine live cells. Washed cells were fixed with 2% formaldehyde-PBS and permeabilized in 3.0% Triton X-100. Cells were treated with Fc blocker (BD 564220) in a mixture of Brilliant Stain Buffer (BD 563794) and eBioScience Flow Staining buffer (Invitrogen 00-4222-26). Cell staining was performed using antibody-fluorophore conjugates and, in parallel, with isotype IgG conjugates of corresponding fluorophores (BD Biosciences). Stained cells were subjected to data acquisition using a Stratedigm S1400Exi flow cytometer. Data were analyzed by FlowJo software. Compensation was performed using UltraComp eBeads Plus Compensation Beads (Invitrogen 01-3333-41) for each IgG-fluorochrome conjugate.

Tissue culture supernatants and sera were analyzed using a custom-made human Magnetic Luminex Assay Kit (LXSAHM-15; R&D Systems) for the following analytes: TNF-α, IFNα, IFNβ, IFNγ, IL-1β, IL-2, IL-4, IL-6, IL-8, IL-10, IL-17, CCL2, M-CSF, GM-CSF, and VEGF-A. Samples were processed in replicates and quantified using the BioPlex 200 System (Bio-Rad). The concentration of each analyte was expressed as pg/mg of total protein. Analyte levels in normal serum or control supernatants were set at 100% when compared to test samples.

### Statistical Analysis.

Statistical analyses were performed using Prism Graphpad software 9.0. Comparison between groups was made using one-way ANOVA. Multiple comparisons were done using the Šidák test with a single pooled variance. Cytokine/chemokine/growth factor data are presented as mean±SEM. P values are expressed as: P≥0.05 (ns), P<0.05 (*), P<0.01 (**), P<0.001 (***), and P<0.0001 (****). A P value of 0.05 or lower was considered statistically significant.

### Results

DCLK1 is expressed at the site of SARS-CoV-2 infection in the lung and liver of COVID-19 patients.

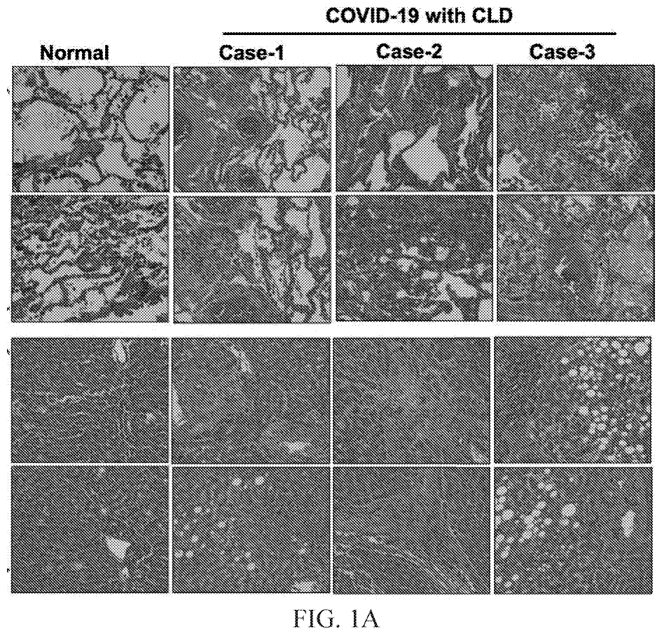

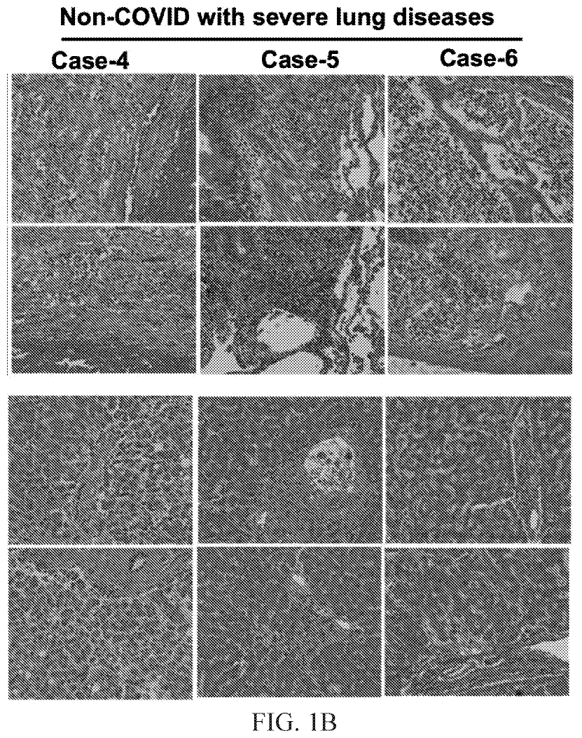

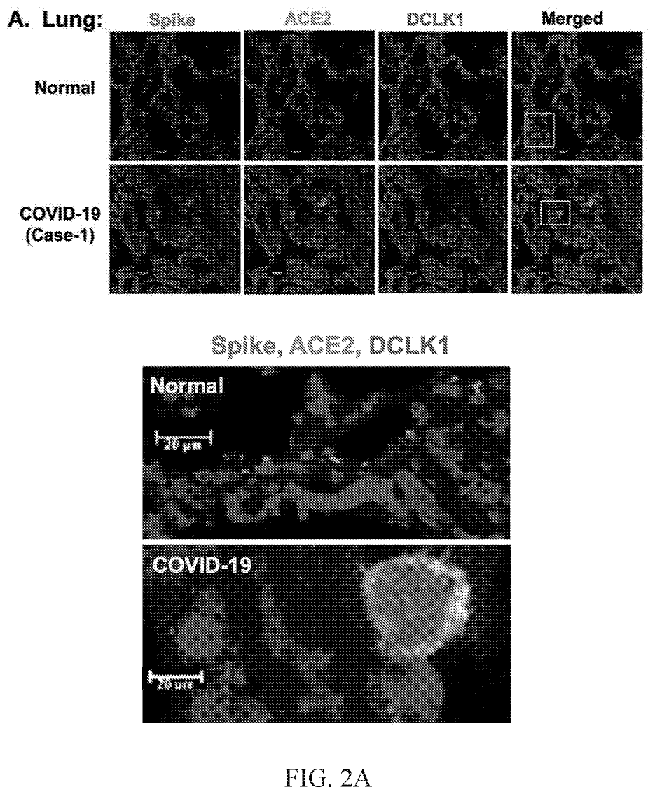

Lung and liver tissues of individuals (n=11) who were autopsied by the Oklahoma City Office of Chief Medical Examiner (OCME) during the early pandemic were analyzed. Based on detailed autopsy investigations and H&E staining results (33), the subjects were identified as: (i) COVID-19 with CLD (Cases 1, 2, and 3); (ii) non-COVID-19 (SARS-CoV-2-negative) with severe lung disease but no liver disease (Cases 4, 5, and 6); and (iii) COVID-19 with mild liver disease or histologically normal liver (Cases 7, 8, and 9). For comparison, we used as controls the autopsies of two persons (N-1 and N-2) who did not have COVID-19 or lung or liver pathology but died from drowning or cardiomyopathy. The clinical findings and representative photomicrographs of the lung and liver histology and histopathology for these cases and controls are shown in FIGS. 1A-1B and summarized in Table 1. Immunofluorescence staining of tissues for the COVID-19 cases with CLD (Cases 1, 2, and 3) showed strong patchy staining for ACE2, Spike protein of SARS-CoV-2, and DCLK1, in both lung (FIG. 2A, only Case 1 is shown) and liver (FIG. 2B). The COVID-19 cases with mild or no steatosis (Cases 7, 8, and 9) showed absent to moderate staining for ACE2, Spike, and DCLK1 in lung and rare to no staining in liver. In contrast, lungs and livers from normals (N-1 and N-2) and non-COVID-19 cases with pre-existing lung disease (Cases 4, 5, and 6) showed minimal to no staining for ACE2 and DCLK1 (FIGS. 1A, 1B, 2A, 2B, Table 1). These results indicated that individuals with CLD who succumbed to SARS-CoV-2 infection had markedly increased expression for ACE2, Spike, and DCLK1 in lung and liver compared to COVID-19 cases without CLD and to non-COVID-19 controls.

TABLE 1
Summary of autopsied subjects with immunohistochemical (IHC) findings for lung and liver^{#}

| | Age | | Lung | Lung IHC | | | | Liver | Liver IHC | | | |
|---|---|---|---|---|---|---|---|---|---|---|---|---|
| Autopsied subjects | (y) | Gender | histopathology | ACE2 | Spike | DCLK1 | S100A9 | histopathology | ACE2 | Spike | DCLK1 | S100A9 |
| Normal lung and liver | | | | | | | | | | | | |
| Normal (N)-1 | 22 | F | Normal | + | − | + | + | Normal | − | − | +/− | + |
| Normal (N)-2 | 28 | F | Normal | + | − | + | + | Normal | − | − | − | + |
| COVID-19 with chronic liver disease | | | | | | | | | | | | |
| Case-1 | 77 | M | Diffuse alveolar damage | +++ | +++ | +++ | +++ | Pre-existing centrilobular steatosis | +++ | +++ | ++ | +++ |
| | | | | | | | | and portal inflammation with focal | | | | |
| | | | | | | | | necrosis and acute inflammation | | | | |
| Case-2 | 42 | M | Acute broncho- | +++ | +++ | +++ | +++ | Pre-existing micronodules with | ++ | +++ | ++ | +++ |
| | | | pneumonia with bilateral | | | | | bridging fibrosis and chronic | | | | |
| | | | consolidations | | | | | inflammation of portal tracts | | | | |
| Case-3 | 26 | M | Diffuse alveolar damage | + | +++ | +++ | +++ | Pre-existing advanced steatosis | ++ | +++ | +++ | +++ |
| | | | | | | | | with microthrombi in portal arteries | | | | |
| Non-COVID-19 with severe lung disease | | | | | | | | | | | | |
| Case-4 | 53 | M | Diffuse alveolar damage | ND | ND | + | + | Pre-existing mild steatosis with mild | ND | ND | − | +/− |
| | | | die to exposure to toxic | | | | | chronic inflammation of portal tracts | | | | |
| | | | fumes | | | | | | | | | |
| Case-5 | 47 | M | Aspiration pneumonia | ND | ND | + | + | Pre-existing chronic inflammation of portal tracts | ND | ND | − | +/− |
| | | | due to drowning | | | | | | | | | |
| Case-6 | 74 | M | Diffuse alveolar damage | ND | ND | − | − | Pre-existing chronic inflammation of | ND | ND | + | + |
| | | | due to necrotizing | | | | | portal tracts | | | | |
| | | | bacterial | | | | | | | | | |
| COVID-19 with normal or mildliver disease | | | | | | | | | | | | |
| Case-7 | 53 | M | Acute bacterial | ++ | ++ | ++ | + | Normal | +/− | +/− | − | + |
| | | | pneumonia with diffuse | | | | | | | | | |
| | | | alveolar damage, intra- | | | | | | | | | |
| | | | alveolar edema, and | | | | | | | | | |
| | | | microthrombi | | | | | | | | | |
| Case-8 | 36 | M | Diffuse alveolar damage | + | + | +/− | +++ | Pre-existing mild steatosis | − | − | − | + |
| | | | with microthrombi | | | | | | | | | |
| Case-9 | 52 | F | Early changes of viral | − | + | − | ++ | Pre-existing mild steatosis | − | − | − | + |
| | | | pneumonia | | | | | | | | | |

^{#}Staining was assessed as being absent (−), rare (+/−), weak (+), moderate (++), or strong (+++)

M2-like macrophages co-express DCLK1 and S100A9 and correlate with disease severity.

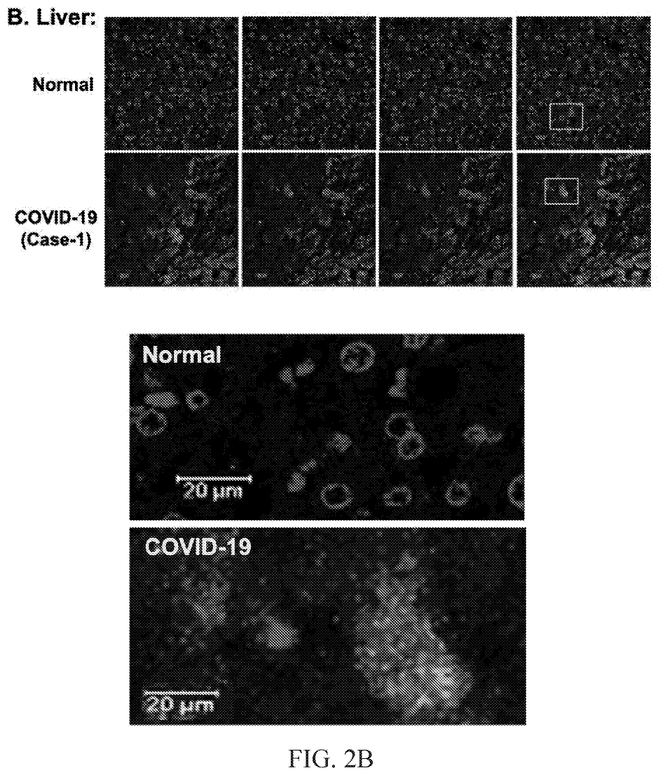

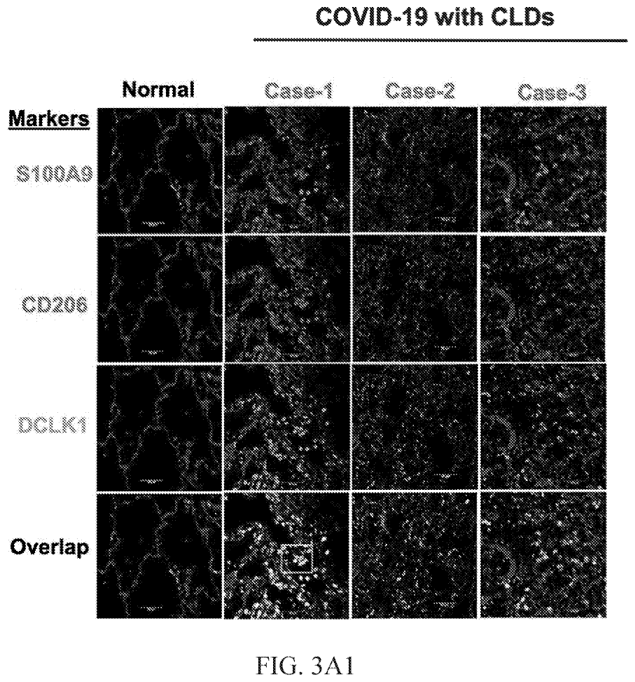

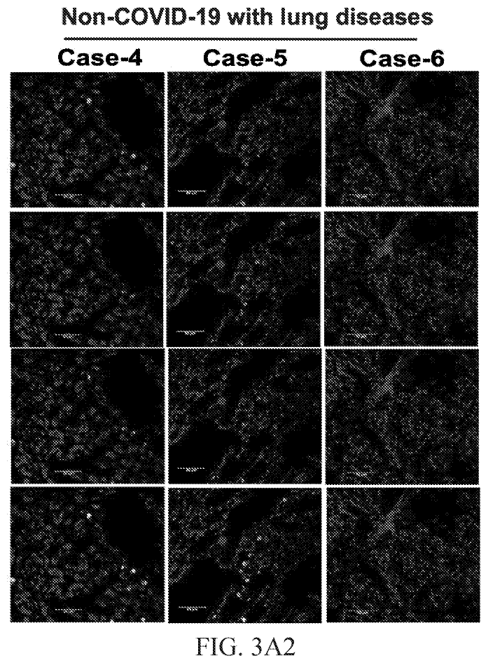

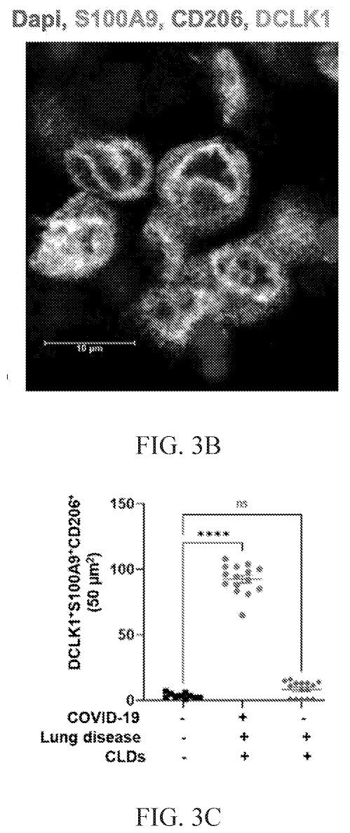

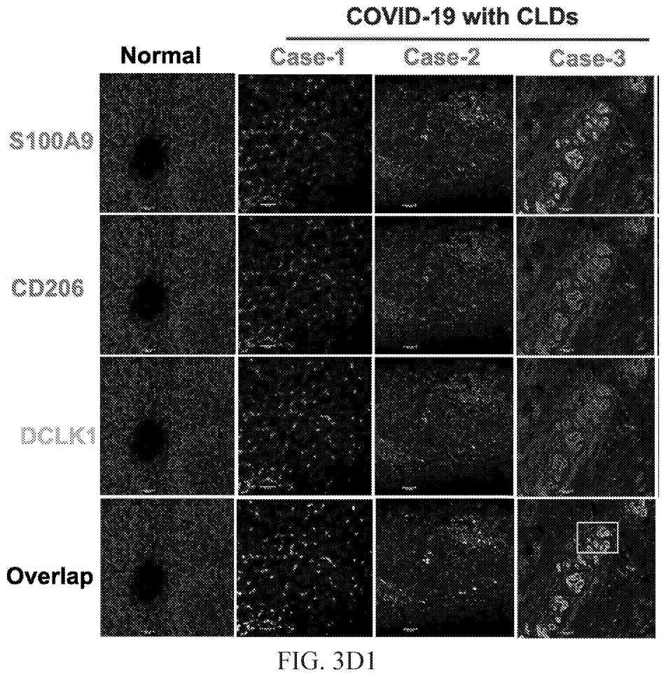

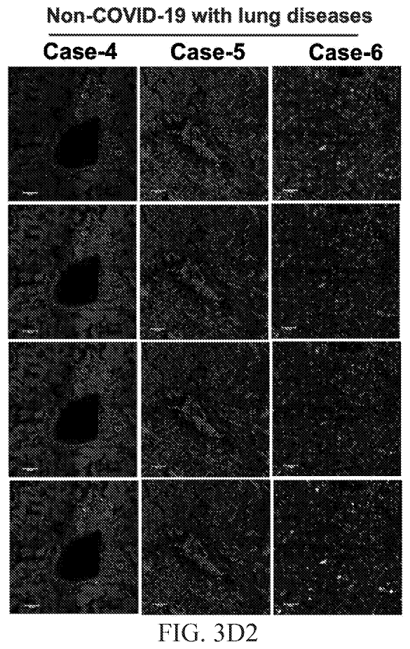

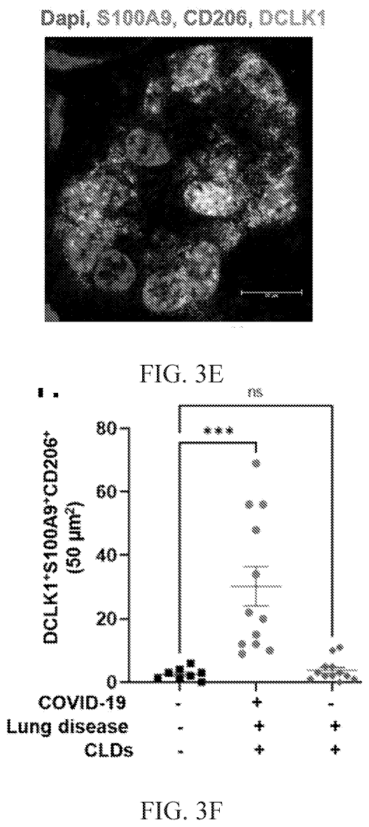

S100A9 plays important roles in dysregulation of innate immune system in many diseases including COVID-19. It is also positively regulated by DCLK1. We observed strong S100A9 staining in both epithelial, alveolar and sinusoidal cells during initial investigations, (Cases 1, 2, and 3; Table 1), This observation led us to determine DCLK1 and S100A9 status in immune cells. We co-stained for DCLK1, S100A9, and macrophage markers. The lungs of COVID-19 cases with CLD showed extensive co-staining for DCLK1 and S100A9 in CD206⁺ M2-like polarized macrophages (FIGS. 3A1,3A2,3B). These triple-positive cells (DCLK1⁺S100A9⁺CD206⁺) were primarily located in alveolar spaces and adjacent to interstitial compartments. Quantitative evaluation of 5 areas within stained lung tissues for each individual revealed that COVID-19 cases with CLD had 4-5 fold increased numbers of the triple-positive macrophages compared with non-COVID-19 cases with lung disease (Cases 4, 5, 6, FIG. 3C). Similarly, high levels of DCLK1⁺S100A9⁺CD206⁺ macrophages were also observed in the livers of Cases 1-3 compared to Cases 4-6. (FIGS. 3D1,3D2,3E,3F). However, staining intensity was minimal and numbers of triple-positive cells were rare in lungs and livers of normal (N-1 and N-2) and non-COVID-19 subjects (Cases 4-6) and COVID-19 subjects without CLD (Cases 7-9). For the latter group, S100A9⁺/CD68⁺ M1-like macrophages were observed instead of triple-positive cells (not shown). The triple-positive cells had kidney-shaped nuclei that were morphologically compatible with macrophages (FIG. 3B). In addition, a small number of triple-positive N2-like neutrophils were also noted, which were defined by their characteristic hypersegmented nuclei and CD206 expression. These findings suggest that increased numbers of triple-positive DCLK1⁺S100A9⁺CD206⁺ M2-like macrophages and N2-like neutrophils are common in lung and liver of COVID-19 subjects with CLD.

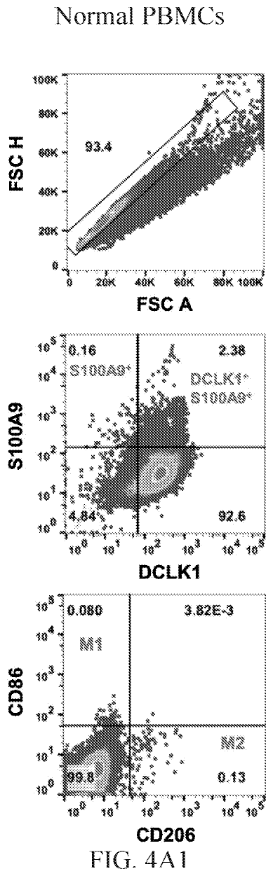

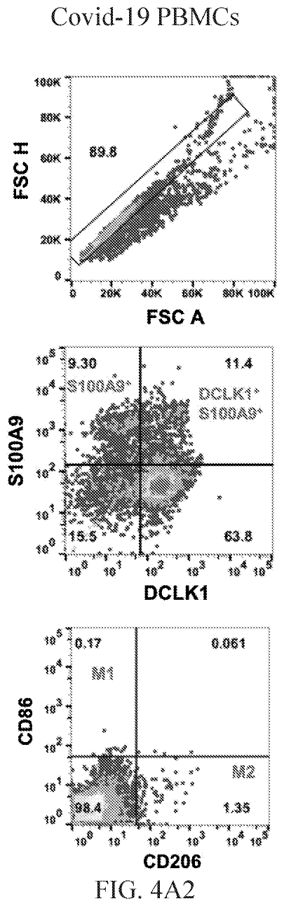

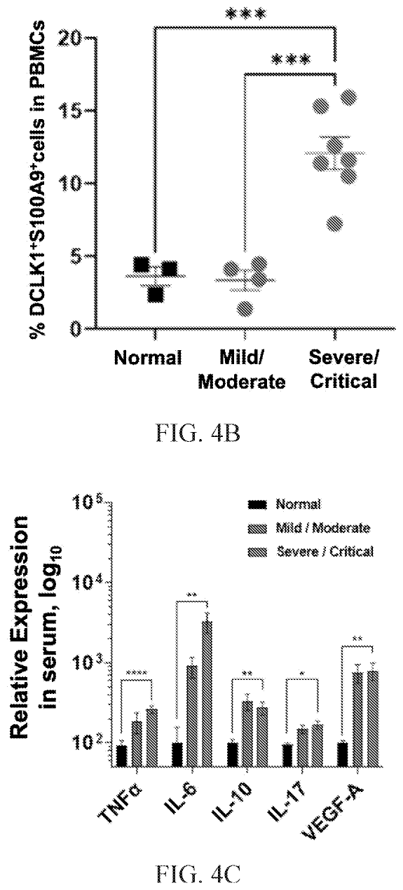

We next wanted to determine whether triple-positive immune cells also occurred as circulating cells in blood of COVID-19 patients. Because PBMCs were not available for post-mortem cases, we analyzed PBMCs from the blood of 11 hospitalized COVID-19 patients (4 with mild-to-moderate disease and 7 severe/critical disease) and from the blood of 3 normal healthy adults. We note that these patients were hospitalized in 2020 and received standard of care for COVID-19 at that time (e.g., remdesivir, steroids, and/or convalescent plasma). PBMCs of severe COVID-19 patients had 3- to 4-fold greater numbers of circulating DCLK1⁺S100A9⁺ mononuclear cells compared to PBMCs from normals or patients with mild-to-moderate COVID-19 (FIGS. 4A1,4A2,4B). Few of these cells, however, had CD86⁺ M1-like or CD206⁺ M2-like phenotypes indicative of polarized macrophages. Analysis of sera for COVID-19 patients revealed high levels of pro-inflammatory (TNF-α, IL-6, and IL-17) and anti-inflammatory (IL-10) cytokines, and the angiogenic factor VEGF-A compared to normal control sera (FIG. 4C).

DCLK1 and S100A9 inhibitors block production of infectious SARS-CoV-2 particles and inflammatory responses.

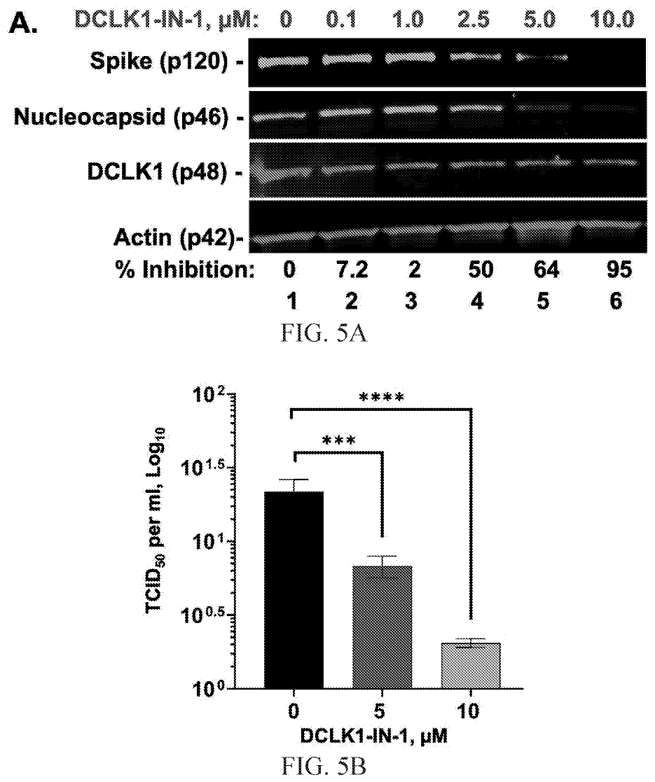

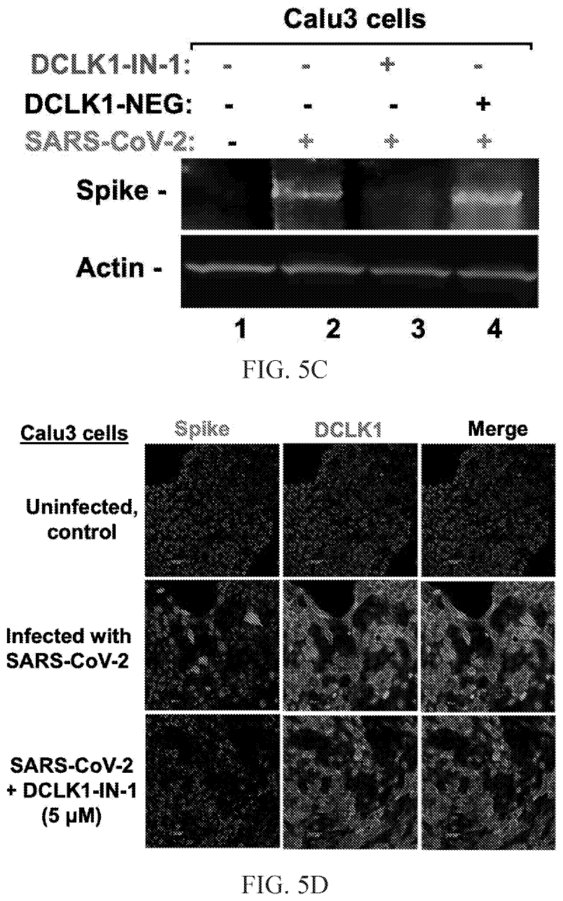

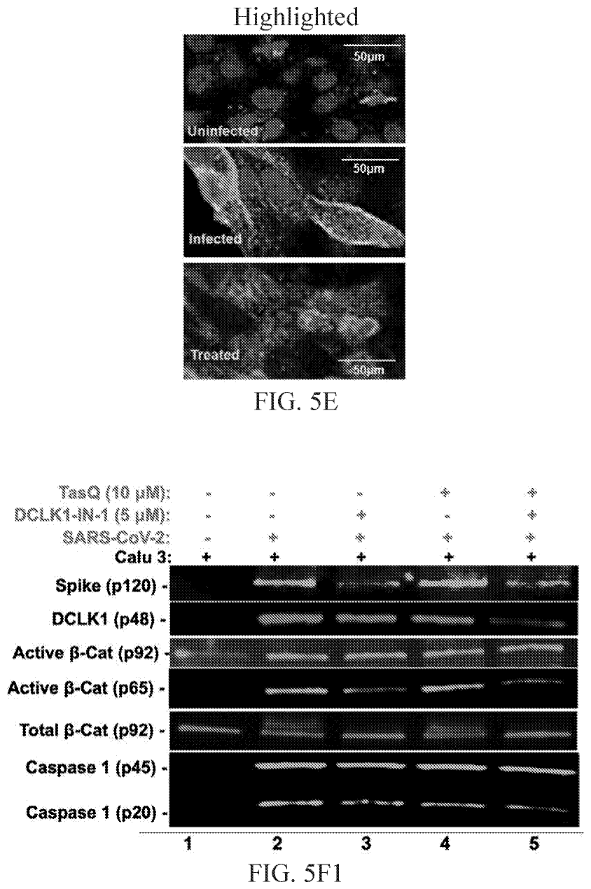

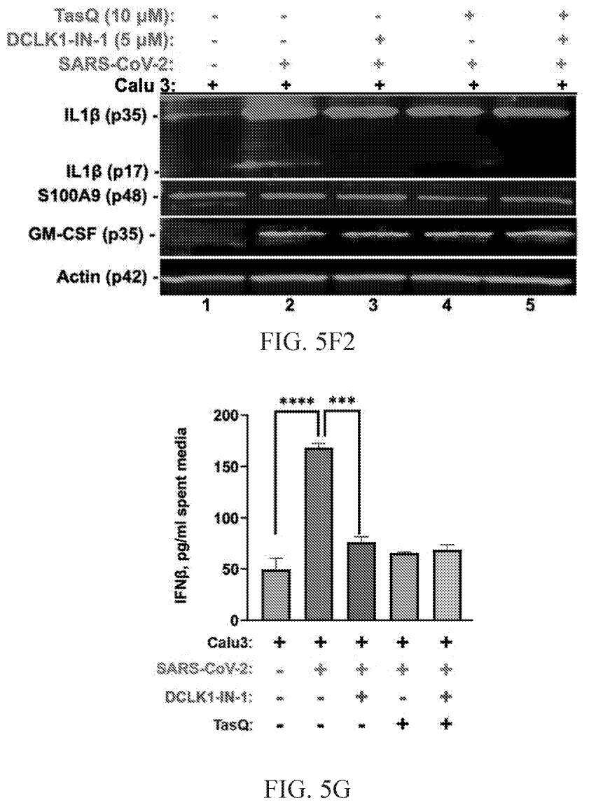

To further explore the potential role of DCLK1 in SARS-CoV-2 infection, we infected Calu3 cells (a lung adenocarcinoma cell line) and measured production of infectious virus particles following treatment with a well-characterized small-molecule inhibitor of DCLK1 kinase (DCLK1-IN-1). This inhibitor strongly binds the kinase domain of DCLK1 with high specificity (K_{D}=109 nM). A structural analog, DCLK1-NEG, with low binding affinity was used as a negative control. The DCLK1-IN-1 treatment of infected cells showed a strong dose-response reduction in intracellular viral proteins (nucleocapsid and Spike) (FIG. 5A, lanes 4-6) compared to untreated control (lane 1). There was an approximate 65% and 95% loss of these proteins at 5 μM and 10 μM, respectively. DCLK1 protein levels were not significantly affected by treatment. These results suggested that the kinase activity of DCLK1 promoted viral replication. TCID₅₀ analysis of SARS-CoV-2 infectious particles in culture supernatants from treated and untreated samples showed a 4-fold and 10-fold decrease in infectivity at 5 μM and 10 μM DCLK1-IN-1, respectively (FIG. 5B). The negative control DCLK1-NEG at 5.0 μM did not reduce Spike (FIG. 5C), suggesting that reductions in SARS-CoV-2 viral particle production by DCLK1-IN-1 was specifically due to inhibition of kinase activity. We confirmed DCLK1-IN-1-mediated downregulation of Spike using immunofluorescence (FIG. 5D, lower panels; FIG. 5E, highlighted in lower panel). It was notable that many cells with little to no staining for Spike showed intense staining for DCLK1 implying potential paracrine signaling between cells.

To further investigate mechanisms of DCLK1 regulation of SARS-CoV-2 pathogenesis, we next treated infected Calu3 cells with DCLK1-IN-1 alone or in combination with tasquinimod (TasQ), an S100A9 inhibitor. SARS-CoV-2 induction of DCLK1 was accompanied by activation of the full-length (p92) and a smaller form (p65) of active β-catenin (FIG. 5F1-5F2, lane 2 compared with lane 1). An anti-active β-catenin monoclonal antibody that specifically recognizes the unphosphorylated N-terminus motif was used for detecting these bands. DCLK1-IN-1 plus tasquinimod attenuated activate β-catenin(p65) and DCLK1 (FIG. 5F1-5F2, lane 5), suggesting induction of DCLK1/S100A9 through β-catenin (p65) by SARS-CoV-2. Similarly, viral infection increased p45 (unprocessed) and p20 (active) forms of caspase 1 (FIG. 5F1-5F2, lane 2). This correlated with high levels of pro-IL-1β (p35) and active (p17) IL-1β (a downstream target of caspase 1) and GM-CSF. DCLK1-IN-1, tasquinimod, and combined treatments downregulated active forms of caspase 1 and IL-1β (FIG. 5F1-5F2, lanes 3, 4, and 5). Intracellular GM-CSF and S100A9 levels were not affected by these inhibitors. We next determined whether interferon (IFN) β as part of the cellular antiviral response to SARS-CoV-2 infection was modulated by inhibiting the kinase activity of DCLK1 or S100A9. IFNβ produced by infected cells was normalized when treated with the inhibitors (FIG. 5G). These findings suggest that targeting the kinase activity of DCLK1 and/or S100A9 may be useful to limit inflammation and bolster antiviral cellular responses to SARS-CoV-2.

SARS-CoV-2 infection induces DCLK1⁺S100A9⁺ mononuclear immune cells and M1-like macrophages.

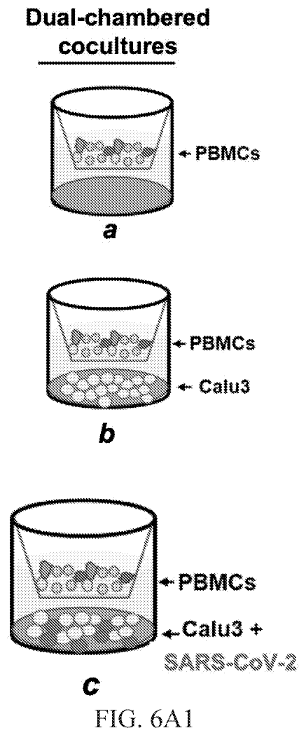

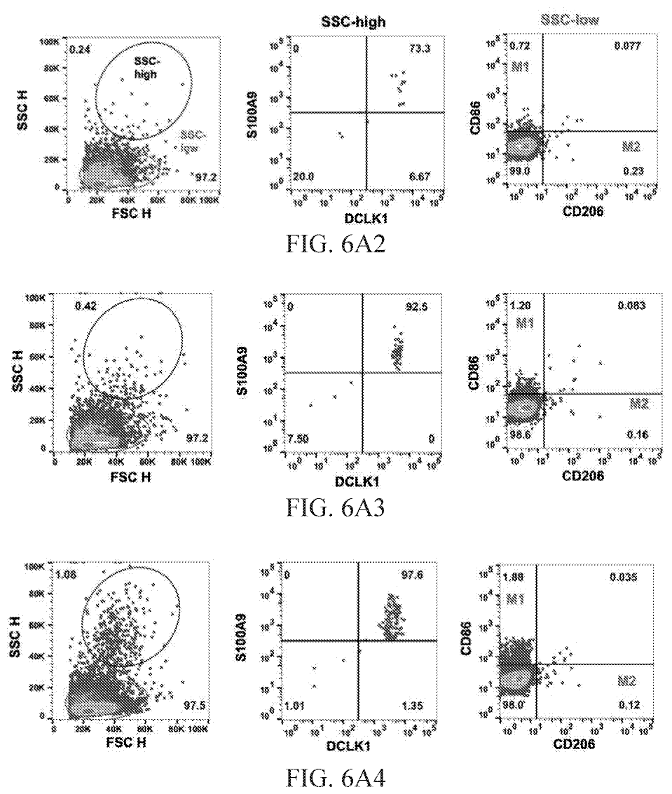

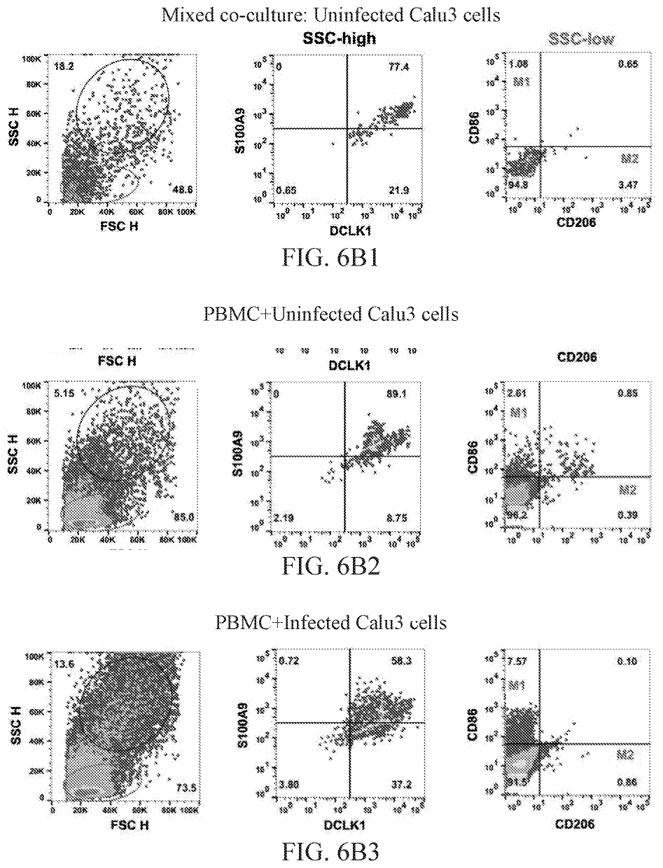

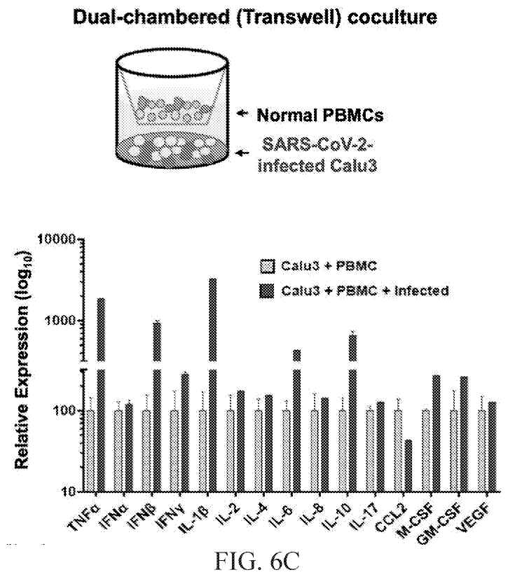

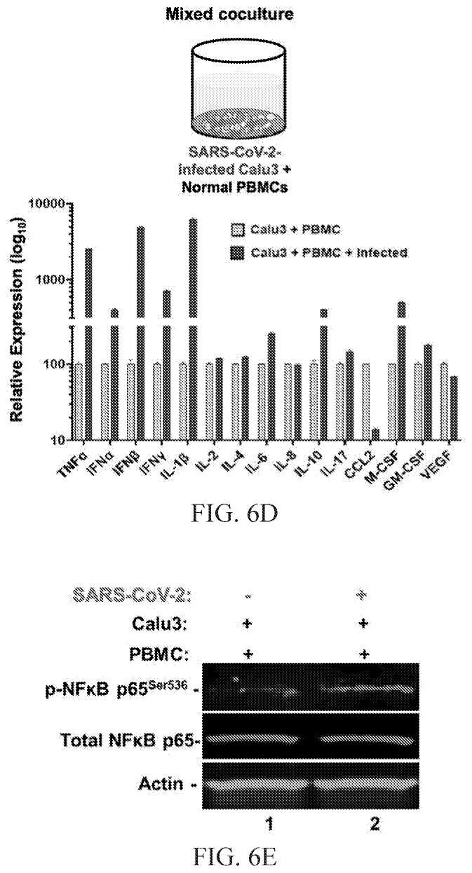

We next determined whether the association of triple-positive DCLK1⁺S100A9⁺CD206⁺ macrophages with severe/critical SARS-CoV-2 infection was due to the exposure of immune cells to viral-infected lung or liver cells. Infected Calu3 cells were incubated with normal human PBMCs in a dual-chamber system that prevented cellular contact but allowed for the free diffusion of soluble factors. When cultured alone, live singlet PBMCs gated for SSC-high and SSC-low morphologies showed few DCLK1⁺S100A9⁺ mononuclear cells and few M1-like (CD86⁺) or M2-like (CD206⁺) macrophages (FIG. 6A1-6A4, upper panels). However, compared to uninfected controls, an increase in DCLK1⁺S100A9⁺ SSC-high cells was observed when PBMCs were exposed to SARS-CoV-2-infected Calu-3 cells (FIG. 6A1-6A4, left bottom panel). These SSC-high cells were predominantly negative for CD86 and CD206 markers, suggesting they were not M1 or M2 polarized macrophages (data not shown). Finally, for PBMCs exposed to infected Calu3 cells, SSC-low cells representing lymphocytes and monocytes showed little to no change in the small proportions of M1-like and M2-like cells compared to untreated controls (FIG. 6A1-6A4, lower right panels). These results showed that soluble factors from SARS-CoV-2-infected cells generated DCLK1⁺S100A9⁺ cells although did not polarize blood monocytes into M1-like or M2-like macrophages.

To determine the potential role of cell-cell contact on induction of DCLK1⁺S100A9⁺CD206⁺ mononuclear immune cells, a similar analysis was performed using PBMCs and SARS-CoV-2-infected Calu3 cells in mixed culture. Again, a substantial increase in DCLK1⁺S100A9⁺ cells was observed in SSC-high populations (FIG. 6B, middle panels). SSC-low cells showed an increase in their proportions of M1-like, but not M2-like cells (FIG. 6B1-6B3, right panels). We tested secreted cytokine/chemokine levels in both coculture formats (transwell and mixed) using spent media. Supernatants showed marked increases in TNF-α, IFNβ, IL-1β, IL-6, and IL-10 for infected cultures compared to uninfected controls in the transwell system (FIG. 6C). In the mixed cocultures that allowed infected cell-immune cell interactions, similar increases were observed in these cytokines along with modest increases in M-CSF, GM-CSF, IFNα, and IFNγ (FIG. 6D). The C-C motif chemokine ligand 2 (CCL2/MCP-1) was down-regulated in mixed culture although increased levels have been reported for patients with severe/critical COVID-19. Since NF-κB is involved in macrophage polarization to the M1-like phenotype, and the upregulation of inflammatory cytokines, we performed Western blots of lysates for the activated form of NF-κB (p-NF-κB(65)^{Ser536}) and total NF-κB in infected and uninfected Calu3 cells cultured with PBMCs (FIG. 6E). While total NF-κB was maintained at basal levels, infection increased the activated form of NF-κB. Since NF-κB is activated by S100A9/TLR4 pathways in macrophages (41), it is likely that increases in activated NF-κB (FIG. 6E lane 2) were due to polarized macrophages. The upregulation of IFNγ and other chemokines along with increased proportions of M1-like macrophages in mixed culture suggests that SARS-CoV-2-infected cells induce a strong pro-inflammatory Th1 response as typically seen in severe COVID-19.

SARS-CoV-2-infected human hepatocytes express DCLK1 and S100A9 and generate a pro-inflammatory immune response.

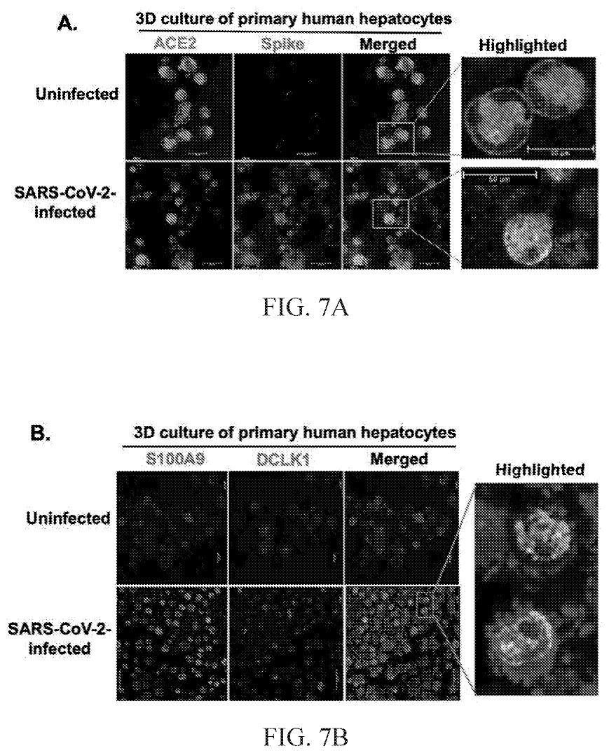

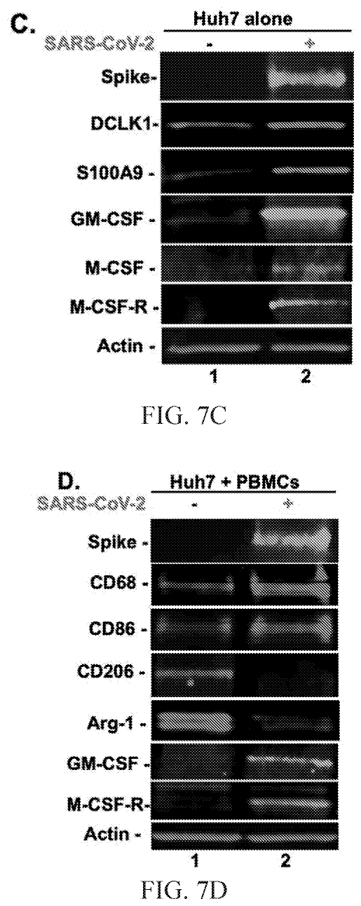

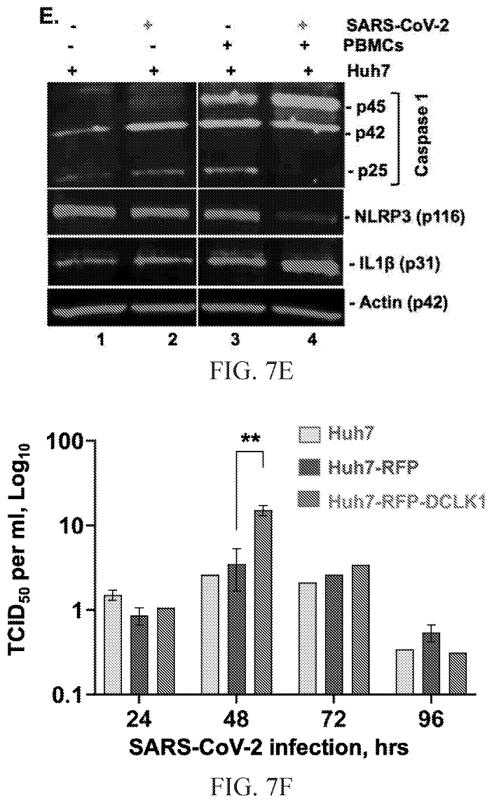

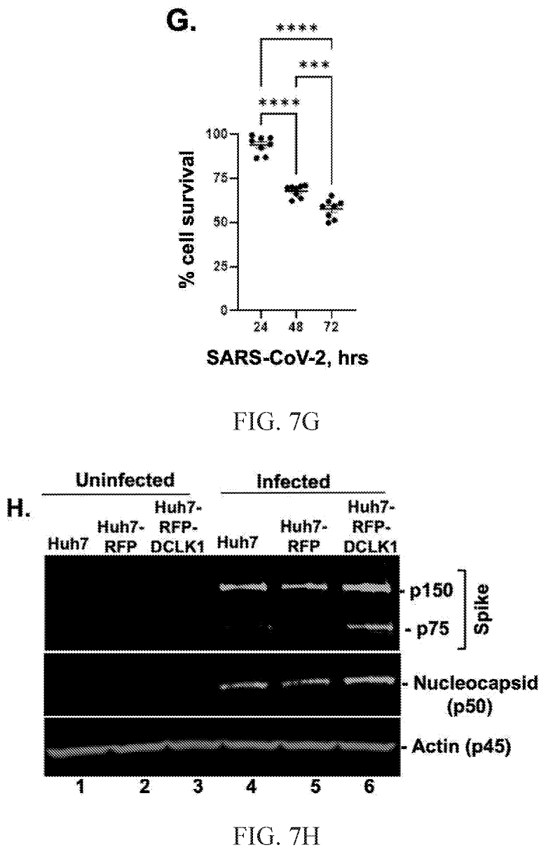

Persons with CLD have a higher risk for fatal outcomes with COVID-19 compared to those without liver disease. Both DCLK1 and S100A9 are highly expressed in CLD, including fatty liver, cirrhosis, and hepatocellular carcinoma. To determine whether SARS-CoV-2-infected cells with high DCLK1 expression have increased viral replication, we used primary human hepatocytes (PHHs) cultured cells on thin Matrigel layers to retain plasticity and lineages. The ACE2 viral receptor was highly expressed under these conditions (FIG. 7A, upper panels). Cells infected with SARS-CoV-2 at a multiplicity of infection of 1 were markedly positive for Spike (FIG. 7A, lower panels). Viral infection also strongly induced DCLK1 and S100A9 (FIG. 7B). As we observed with Calu3 cells and in livers from COVID-19 cases with CLD, ACE2-expressing hepatocytes were susceptible to SARS-CoV-2 and infection was associated with DCLK1 and S100A9 expression.

We next confirmed the susceptibility of Huh7 human hepatoma cells to infection with SARS-CoV-2. Similar to experiments using PHHs, these cells strongly induced DCLK1 and S100A9 when infected (FIG. 7C). SARS-CoV-2 also led to the expression of immunomodulatory cytokines (GM-CSF and M-CSF) and M-CSF-R/CD115 receptors. These cofactors help drive pro-inflammatory signaling and macrophage recruitment/polarization. Mixing SARS-CoV-2-infected Huh7 cells with normal human PBMCs strongly induced a CD68⁺/CD86⁺ M1-like macrophage phenotype with a corresponding loss of CD206⁺/Arg-1⁺ M2-like markers (FIG. 7D). Thus, SARS-CoV-2 infection of DCLK1-expressing hepatoma cells promoted an M1-like polarization of PBMCs. These findings are consistent with observations that used infected Calu3 cells (FIG. 6B1-6B3). Infected Huh7 cells also had increased levels of caspase 1 and IL1β (FIG. 7E). Mixed cultures of infected Huh7 cells with PBMCs led to an accumulation of unprocessed (i.e., high molecular weight) caspase 1 (p45 and p42) and pro-IL-1β (p31) at 48 h. This was accompanied by downregulation of NLRP3. Longer incubations of these cultures were not feasible due to increasing cell death over time (FIG. 7G).

DCLK1 amplifies SARS-CoV-2 production by liver cells.

To test whether DCLK1-overexpressing liver cells generally present in CLD could amplify SARS-CoV-2 production, Huh7 cells were engineered to overexpress N-terminal red fluorescent protein (RFP) tagged human DCLK1 (Huh7-RFP-DCLK1). The biological activities of the expressed RFP-DCLK1 in these cells have been established previously. Huh7 cells and Huh7 cells expressing RFP alone (Huh7-RFP) were used as controls. After infection with SARS-CoV-2, the spent media from infected cells were assayed by TCID₅₀ to determine titers of live viral particles produced by infected cells. Huh7-RFP-DCLK1 cells increased the production of infectious viral particles by 4-fold at 48 h compared to controls (P=0.002; FIG. 7F). With longer incubations (72 and 96 h post-infection), decreases in viral particles were most likely due to cell death (FIG. 7G) and reduced stability of viral particles in spent culture media. Others have reported significant losses of SARS-CoV-2 viability in liquid medium at 37 C within 1-2 days (44). Huh7-RFP-DCLK1 lysates showed increases in the large and S2 forms of Spike and nucleocapsid protein (p46) compared to controls (FIG. 7H) at 48 h, helping substantiate DCLK1-mediated upregulation of viral production (FIG. 7F). These findings indicated that the increased expression of DCLK1 amplified SARS-CoV-2 production in liver cells.

Inhibition of DCLK1 kinase attenuates the SARS-CoV-2-induced cytokine signature.

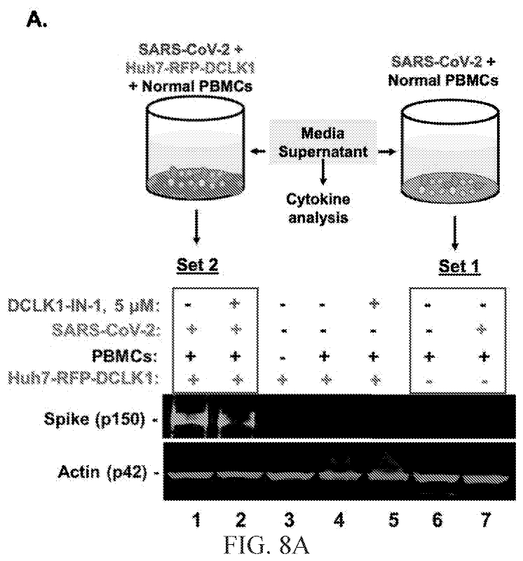

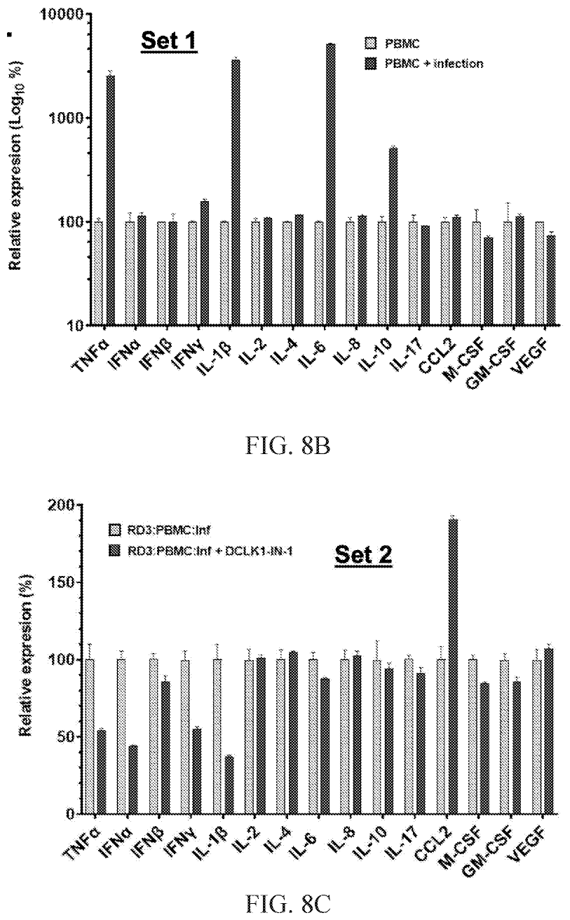

To assess the effect of DCLK1 kinase on cytokine production during SARS-CoV-2 infection, we mixed infected Huh7-RFP-DCLK1 cells with normal human PBMCs in the presence of 5 μM DCLK1-IN-1 for 48 h. Spent supernatants were assayed for cytokines/chemokines/growth factors using a multiplex system. As with infected Calu3 cells, DCLK1-IN-1 blocked Spike (FIG. 8A, lane 2, Set 2). Interestingly, PMBCs were not infected by SARS-CoV-2 and did not produce detectable Spike (FIG. 8A, lane 7, Set 1). However, PBMCs were responsive to SARS-CoV-2-infected cells as indicated by increased secretion of TNF-α, IL-1β, IL-6, and IL-10 in spent media compared to uninfected cells (FIG. 8B). DCLK1-IN-1 treatment led to downregulation of cytokines involved in inflammation and viral response (TNF-α, IFNα, IFNγ, and IL-10; FIG. 8C). Interestingly, CCL2 (monocyte chemoattractant protein-1, MCP-1) was a noteworthy exception as this chemokine showed increased expression when infected cells were treated with DCLK1-IN-1. Since CCL2 participates in normal wound healing, an increase in this chemokine may contribute to tissue healing after viral clearance. In general, this in vitro system mimicked cytokine signatures observed in severe COVID-19 and the DCLK1 kinase inhibitor markedly attenuated this signature.

### II.

### Potential Mechanisms of DCLK1-IN-1-Mediated Inhibition of SARS-CoV-2 Pathology.

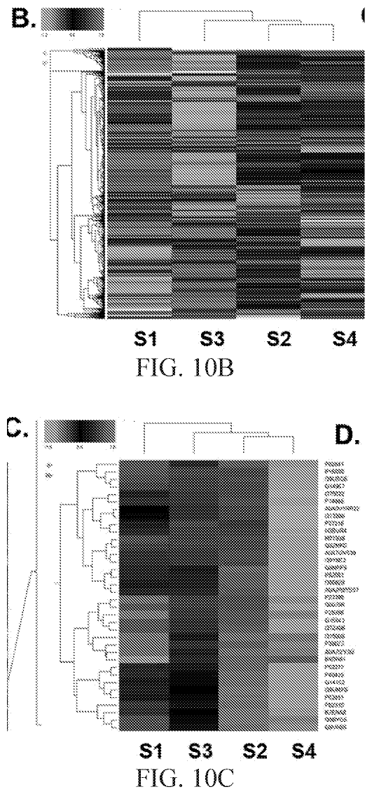

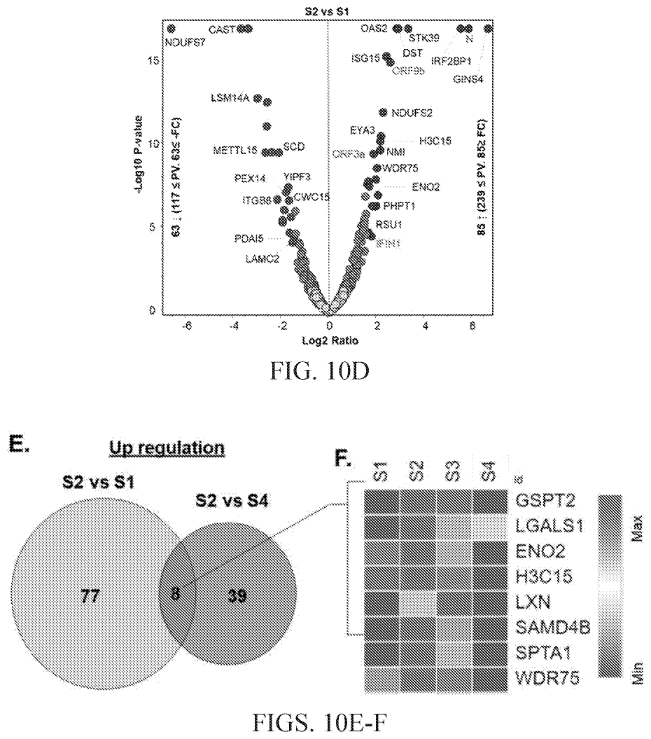

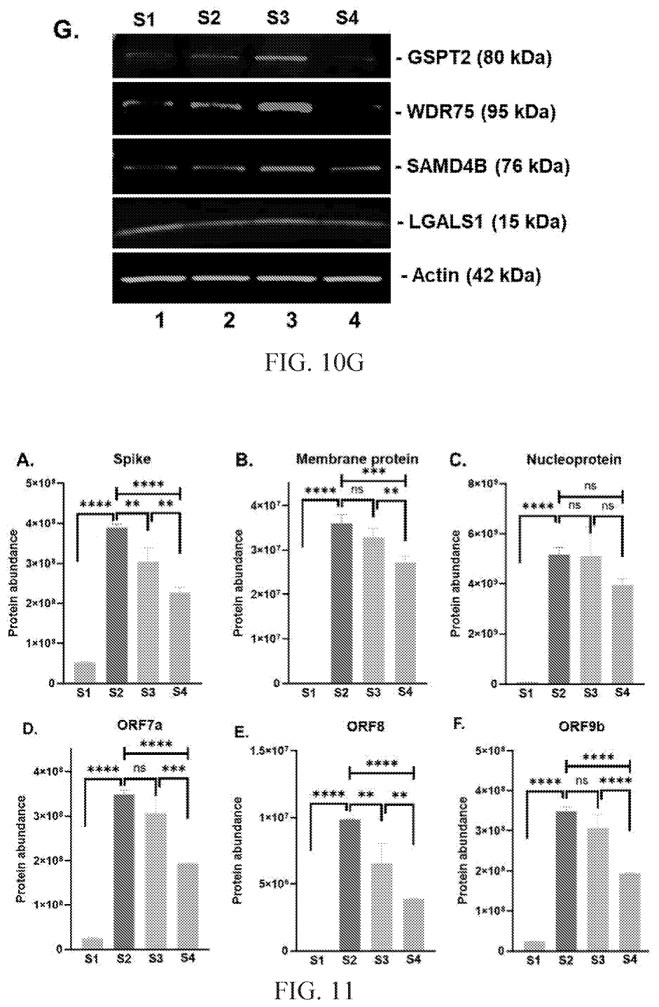

To understand the underlying mechanisms of DCLK1-regulated SARS-COV-2 pathology, we performed label-free quantitative proteomic analysis of SARS-CoV-2 infected Calu-3 lung cells (Sample 2, a.k.a., S2). Cells were treated with dimethyl sulfoxide (DMSO) as the vehicle (Sample 3, a.k.a., S3) or DCLK1-IN-1, a potent small molecule inhibitor of DCLK1 kinase (Sample 4, a.k.a., S4). Uninfected Calu3 cells were used as a baseline control (Sample 1, a.k.a., S1). Each culture was carried out in triplicate. Protein samples (2 g) were analyzed by quantitative proteomic analysis. A total of 18,940 unique peptides corresponding to 3,257 proteins were identified and quantitated for differential protein expression. Principal component analysis (PCA) of total protein abundance showed good separation for each of the four experimental conditions (S1, S2, S3 and S4) as well as high quantitative reproducibility between replicates. (FIG. 10A). Statistical evaluation of these data identified differential expression of host proteins in response to viral infection and DCLK1-IN-1 treatment. Heat map clustering showed 77 proteins were differentially altered by viral infection (FIGS. 10 and 10EF). A subset of SARS-CoV-2-regulated protein clusters (S2, red panel) was restored to normal level (S1, green panel) by DCLK1-IN-1 (S4) but not by DMSO (S3) (FIG. 10C). Volcano plot revealed significant differentially expressed proteins in infected Calu3 cells compared to uninfected cells (S2 vs. S1, FIG. 10D). Venn diagram and heat map analyses identified 8 host proteins that were significantly upregulated by SARS-CoV-2 (GSPT2, LGALS1, ENO2, H3C15, LXN, SAMD4B, SPTA1 and WDR75). These proteins normalized after treatment with DCLK1-IN-1 (lane 4, FIG. 10EF). Proteomic results for GSPT2, SAMD4B, and WDR75 were validated by Western blot (FIG. 10G). Proteomic data also identified 21 cellular proteins that were downregulated by SARS-CoV-2 infection. Several of these proteins were restored to normal by DCLK1-IN-1 (data not shown). Reactome and KEGG pathway analyses (ShinyGO, version 0.76) suggested activation of pathways for cell-cell communication, NF-κB signaling, interferon-stimulated genes, and cytokine and antiviral responses. Overall, the global cellular response of SARS-CoV-2 infected cells to DCLK1-IN-1 treatment shifted dramatically towards recovery and better mitochondrial protein function indicated by improved translational regulation (data derived from Reactome analysis, not shown).

DCLK1 inhibitor significantly downregulated the expression of viral proteins involved in transcription-replication, RNA packaging, and virion formation.

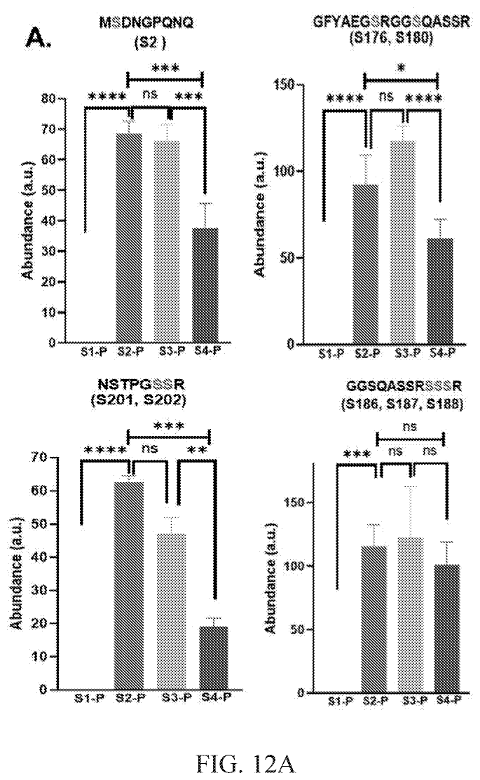

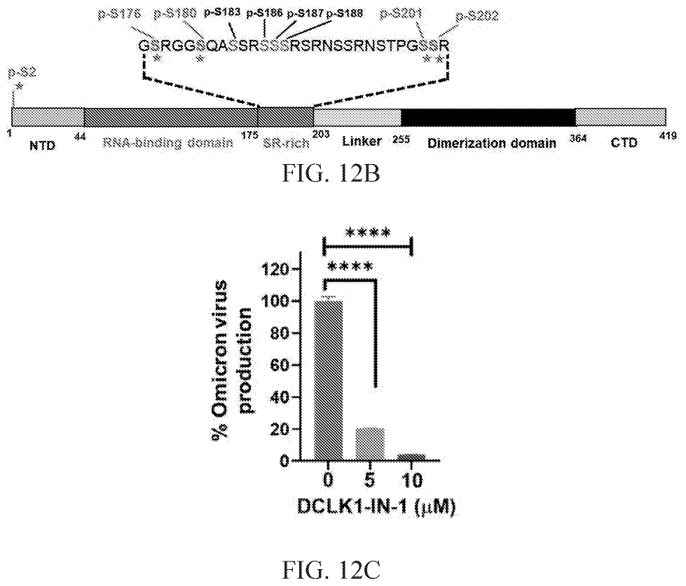

Proteomic analysis revealed that two viral structural proteins (membrane and Spike) and three accessory proteins (ORF7a, ORF8, and ORF9b) were significantly downregulated in the SARS-CoV-2 infected cells that were treated with DCLK1-IN-1 (FIG. 11). However, nucleocapsid protein, as the most abundantly expressed viral protein, was not decreased. These findings suggested that inhibition of DCLK1 kinase downregulates critical viral proteins essential for the production of the new viral particles and their spread, and evasion of the host immune system. Our prior work showed that treatment of SARS-CoV-2 infected cells that were treated with DCLK-IN-1 had significantly decreased production of infectious virions. We speculate that blocking DCLK1 kinase with DCLK1-IN-1 inhibits viral transcription-replication complexes (TRCs). This supposition is further strengthened by our extensive phosphoproteomic analysis described in FIGS. 12A-12B. There are ˜120 phosphorylated sites in SARS-CoV-2 viral proteins (N, M, S, and 3a). The nucleocapsid (N) protein modulates multiple biological processes (e.g., RNA packaging, viral production, and TRCs functions). We detected phosphorylation of serine residues (S2, S23, S79, S176, S180, S183, S186, S187, S188, S201 and S202) in the viral nucleocapsid protein from infected Calu3 cells (FIGS. 12A and 12B, SEQ ID NOS:1-5). DCLK1-IN-1 treatment (S4-P) resulted in a significant decrease in phosphorylation of five serine residues (S2, S176, S180, S201, and S202) compared with untreated (S2-P) and vehicle-treated (S3-P) infected cells. Interestingly, except for S2-P sample, all phosphorylation sites are located in the SR-rich region of the nucleocapsid protein. The DCLK1-responsive serine residues are highly conserved in Omicron lineages currently circulating worldwide.

We determined whether DCLK1-IN-1 successfully inhibits Omicron virus production similar to the original Wuhan strain. TCID₅₀ assay was performed in the media supernatant of Calu3 cells infected with culture-generated Omicron B.1.1.529 virus preparations and treated with DCLK1-IN-1 (5 μM and 10 μM). Untreated and vehicle (DMSO)-treated cultures were used as controls. In comparison to the vehicle-treated control, 80%-90% of Omicron virus production was inhibited by the DCLK1-IN-1 treatment (FIG. 12C). These data strengthen our claim that DCLK1 kinase inhibitor treatment is highly relevant to currently circulating Omicron variants worldwide. The antiviral activities of DCLK1-IN-1 are not compromised by viral evolution, strains, and genetic drifts.

Our phosphoproteomic analysis revealed that the viral membrane protein (M) exhibited significantly reduced phosphorylation at S212, S213 and S214 following treatment with DCLK1-IN-1. The M protein plays a central role in virus replication and assembly. These data clearly suggest that the DCLK1 kinase inhibitor acts against multiple steps of viral replication and transcription processes following infection. Beyond these anti-viral effects, DCLK1-IN-1 also has the potential to normalize cellular signaling induced during COVID-19 and thereby accelerate recovery from the disease.

DCLK1-IN-1 inhibits SARS-CoV-2 replication and improves lung histopathology in a murine model of COVID-19.

DCLK1-IN-1 has shown favorable pharmacokinetic profiles in mice (half-life of 2.09 h) with a maximum tolerated dose (MTD) of 100 mg/kg with no adverse effects or loss of body weight. K18-hACE2 transgenic mice (Jackson Lab, #034860) express human ACE2 protein, the surface receptor for SARS-CoV-2, and have been successfully used as a SARS-CoV-2 infection model. Within 6 days of infection, SARS-CoV-2 elicits a COVID-19-like disease including coagulopathy, lung and hepatic injury, and systemic and local pro-inflammatory responses. To test the effects of DCLK1-IN-1 on SARS-CoV-2 pathology in vivo, K18-hACE2 mice were randomized into four groups (6 females and 6 males per group) as follows: Group 1: Infected, untreated; Group 2: Infected, given vehicle alone (DMSO); Group 3: Infected, treated with DCLK1-IN-1 (10 mg/kg body weight, i.p.) daily for 4 days; and Group 4: no infection (negative control).

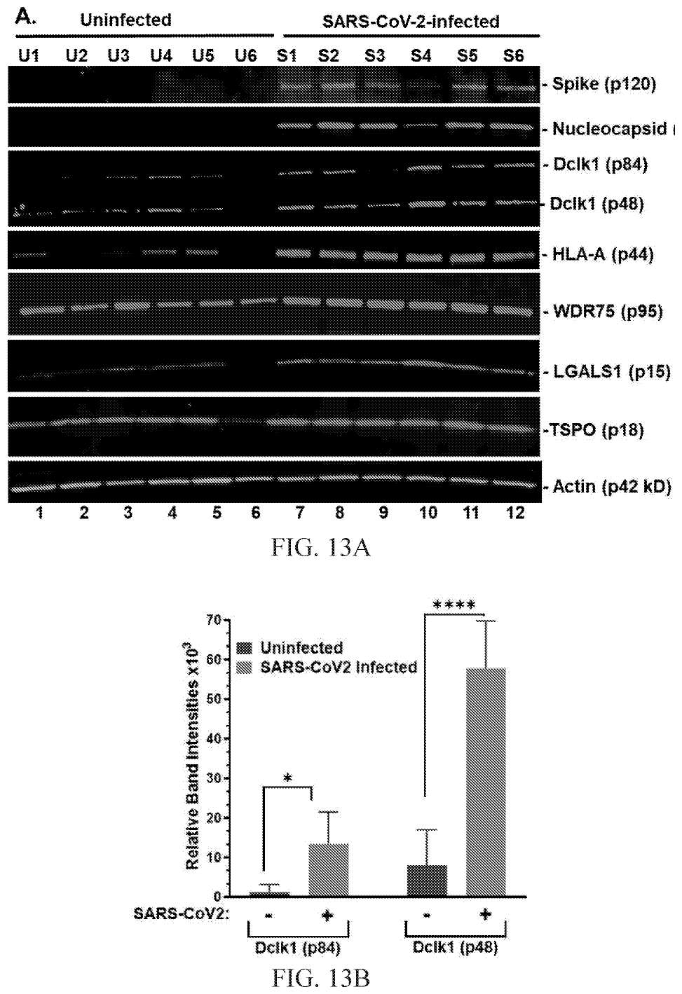

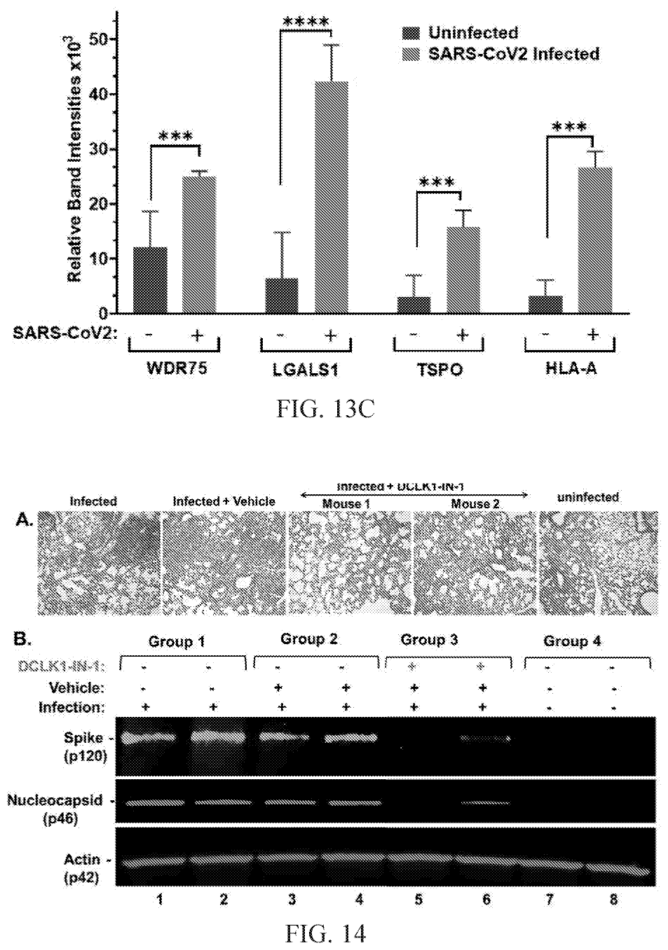

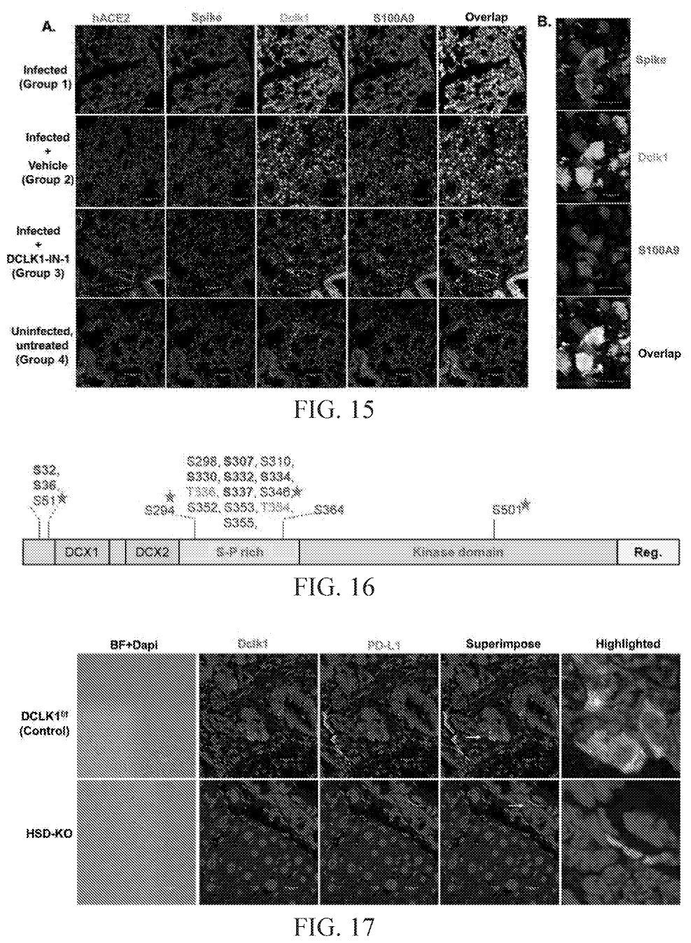

Groups 1, 2 and 3 were infected intranasally (10 μl per nares) with culture-generated SARS-CoV-2 (3×10⁴ plaque-forming units for each mouse, original Wuhan strain). After 6 hours, treatments were initiated as mentioned. Blood, lung, liver, intestine, spleen, and brain were collected on day 5. Western blots of total lung lysates from infected mice showed increased expression of large (3-fold) and small (6-fold) Dclk1 isoforms (FIGS. 13A-13-B). Increased levels of LAGLS1 (Galectin-1, immunosuppressor, and inhibitor of cell growth), WDR75 (ribosome biogenesis), and HLA-A (epitope presentation) were noted in the infected mice (FIG. 13C). These results validated proteomic data from cell culture experiments. H&E stained lung tissues were evaluated. Infected mice (Group 1) and DMSO-treated (Group 2) showed COVID-19-associated pathology including extensive perivascular inflammation, edema, syncytial cells, infiltration of nucleated immune cells (lymphocytes, macrophages, and neutrophils), thrombosis, and necrosis (FIG. 14, upper panel A)). These histopathological features were significantly improved by DCLK1-IN-1 treatment (Group 3, results from two representative mice shown) but not DMSO alone (Group 2). The inhibition of viral replication was supported by reduced or absent expression of Spike and nucleocapsid proteins (FIG. 14, lower panel B; lanes 5 and 6 compared to control lanes 1-4). Confocal microscopy of stained lung tissues showed extensive expression of Spike protein, Dclk1, S100A9, and hACE2 in the infected mice (FIG. 15, Groups 1 and 2). Dclk1 and S100A9 were present in both infected epithelial cells and mononuclear immune cells (FIG. 15 (a-b)). The mononuclear cells were negative for Spike protein. DCLK1-IN-1-treated mice (Group 3) showed rare or weak staining for Spike and low expression of Dclk1 and S100A9. Lung tissue from control mice (Group 4) were negative for Spike and only showed occasional localized epithelial expression of Dclk1 and S100A9. These data suggest a critical role for Dclk1 kinase in COVID-19. We acknowledge that to fully determine any therapeutic potential of Dclk1 kinase inhibitors, or similar drugs, additional studies are needed that can evaluate these agents for potential liver toxicity and to optimize drug dosing.

### Phosphorylation of DCLK1 in SARS-CoV-2 Infected Cells.

We examined publicly available phosphoproteomic data derived from SARS-CoV-2-infected cells. This analysis suggested that DCLK1 is heavily phosphorylated in the protease-sensitive Ser/Pro-rich (SP-rich) region. These modifications likely increase DCLK1 stability by preventing protease attack. This observation provides a putative explanation for the high-level expression of both DCLK1 isoforms in infected Calu3 cells and infected K18-hACE2 mice. Additional phosphorylated residues, including one in the kinase domain (S501) and three in the extreme N-terminus region (S32, S36, S51) were also identified (FIG. 16). Interestingly, both of the DCX regions that bind to tubulin remained unphosphorylated. Recently, it was shown that hyperphosphorylation of DCX domains prevents microtubule-associated protein (MAP) functions.

Liver cells co-express Dclk1 and PD-L1 in chronic liver diseases.

Chronic liver disease (CLD) is a major risk factor for high mortality in COVID-19. The precise reason is not yet fully understood. We previously demonstrated that DCLK1 dysregulates immune responses to SARS-CoV-2 infection in lungs and CLD. This prompted us to develop an HSD-KO (hepatocyte-specific deletion of Dclk1) murine model to better understand Dclk1-regulation of immunosuppressive conditions in the liver during COVID-19. Towards this objective, HSD-KO mice and a parental strain as control (Dclk1^{f/f} with intact Dclk1 expression in hepatocytes) were treated for 16 weeks with DEN/CCl₄ to induce hepatocellular carcinoma (HCC). Livers from these mice were co-stained for Dclk1 and PD-L1 and imaged by confocal microscopy (FIG. 17). Dclk1^{f/f} livers showed Dclk1 at tumor sites but not in normal areas of the liver. A subset of Dclk1^{f/f} cells showed co-expression of PD-L1 (Dclk1⁺PD-L1⁺ phenotype) (FIG. 17, upper panel). We also noticed PD-L1-expressing cells in the vicinity of Dclk1⁺ cells. Furthermore, as expected, Dclk1 expression in hepatocytes at normal and injured sites was absent in HSD-KO mice. Only a few cells in these mice showed PD-L1 staining (FIG. 17, lower panel). These results confirmed that HSD-KO mice did not express Dclk1 in hepatocytes following injury. We also did not observed HCC tumors in their livers. Mice with the intact Dclk1 gene (e.g., Dclk1^{f/f}) developed extensive fibrosis (not shown) and HCC-like tumors that contained Dclk1⁺ and Dclk1⁺PD-L1⁺ cells. These data further reinforce our earlier observations that Dclk1⁺ induces an immunosuppressive microenvironment in injured livers, thereby exaggerating the COVID-19 severity. This highlights the potential importance of DCLK1 as a target for adjunctive therapy in COVID-19, especially for persons with CLD. Using a novel strategy to investigate this hypothesis, we will breed K18-hACE mice that have a knockout of Dclk1 and use these mice to validate DCLK1 involvement in COVID-19 disease severity.

### Discussion

The progression of COVID-19 into severe/critical stages occurs more often in patients with underlying comorbidities such as, for example, chronic liver disease (CLD). Severe/critical SARS-CoV-2 infections are characterized by high levels of viral RNA in plasma, hyperinflammation, and multi-organ failure that results in a increased probability of mortality. The present disclosure demonstrates that DCLK1 plays an important role in SARS-CoV-2 pathogenesis and immune dysregulation in COVID-19, especially for patients with CLD.

We found that DCLK1 was extensively expressed in the lungs and livers of individuals with COVID-19 who had underlying CLD. In vitro DCLK1 increased viral particle production in lung and liver cells by 4-5-fold. A specific DCLK1 kinase inhibitor blunted viral production. This multifunctional kinase is known to modulate the cellular cytoskeleton by phosphorylating tubulin subunits. DCLK1 also autophosphorylates its C-terminus at T688 residue to block hyperphosphorylation of N-terminal doublecortin domains. Hypophosphorylated and unphosphorylated doublecortin domains bind microtubules with higher affinity. Inhibition of T688 phosphorylation by DCLK1-IN-1 can disrupt DCLK1 interactions with microtubules and inhibition of the movement of viral RNA replication complexes. We previously observed the dynamic distribution of DCLK1-microtubule complexes in the filopodia of Huh7 cells by live cell imaging (data not shown). It was recently reported that these protrusions in infected cells contain budding particles of SARS-CoV-2.

The present results indicate the involvement of DCLK1 in a distinctive feed-forward signaling cascade with β-catenin, NF-κB, and S100A9 that can fuel inflammation and immune dysregulation in COVID-19. We found that immune cells localized in alveolar and hepatic sinusoidal spaces in COVID-19 cases with CLD co-expressed DCLK1 and S100A9. Based on CD206 expression, these cells were consistent with M2-like macrophages. We also observed triple-positive DCLK1⁺S100A9⁺CD206⁺ cells with segmented nuclei that likely represent N2-like neutrophils. Few DCLK1⁺S100A9⁺-expressing cells were observed in non-COVID-19 cases with chronic underlying lung disease or in normal controls (Table 1). In hospitalized patients with severe/critical COVID-19, these dually-positive cells were detected in blood. The SARS-CoV-2-dependent generation of these cells was substantiated using dual-chamber and mixed culture systems. These observations are consistent with prior reports showing high levels of calprotectin in the blood of patients with severe COVID-19. The M2- and N2-like phenotypes noted in autopsied COVID-19 cases would be expected to suppress anti-viral innate immune responses, in part, through the upregulation of IL-10.

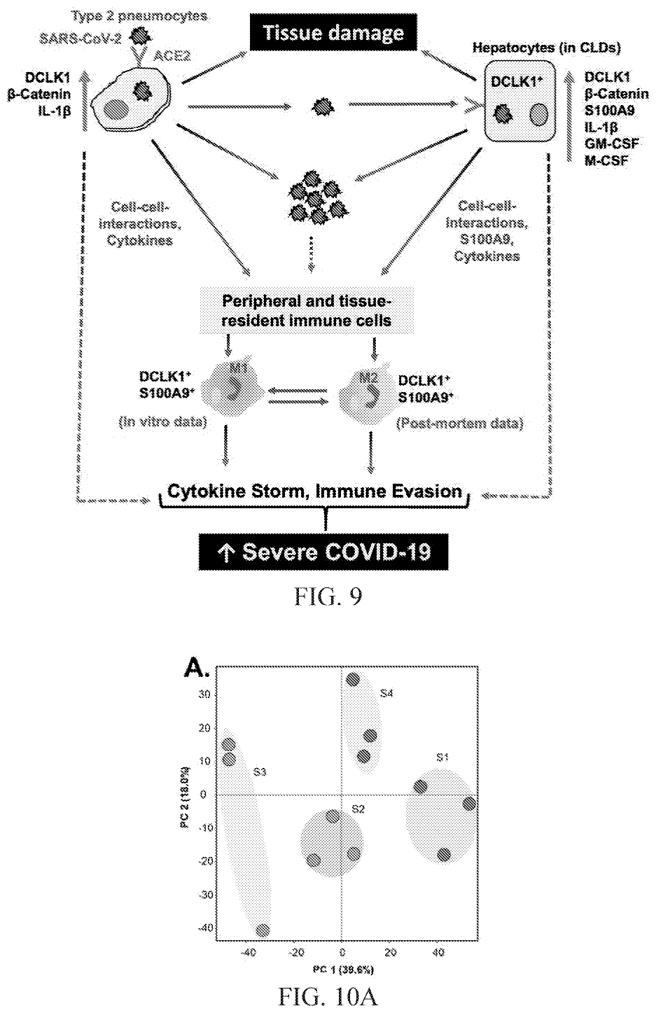

We found that DCLK1 facilitated the production of important inflammatory mediators in vitro. SARS-CoV-2-infected cells also induced GM-CSF, a chemoattractant that helps polarize macrophages toward an inflammatory M1-like phenotype. Without wishing to be bound by theory, in our model, inhibiting DCLK1 kinase blocked inflammatory cytokines (IL-1β, IL-6, and TNF-α). Viral infection of liver cells also led to the induction of M-CSF and its receptor along with IL-10. IL-10 activates immunosuppressive immune cells (Th2) and helps polarize macrophages toward an CD206⁺ M2 phenotype. The induction of the M1 phenotype would enhance inflammatory cytokine production while M2 phenotypes could facilitate higher levels of viremia by suppressing antiviral responses (FIG. 9). The present in vitro studies revealed that targeting both DCLK1 and S100A9 downregulated DCLK1 decreased β-catenin activation, and blocked caspase 1/IL-1β signaling. However, GM-CSF levels were not affected. In a clinical trial of lenzilumab, a GM-CSF inhibitor, the survival of COVID-19 patients was significantly improved. The potential combination of a DCLK1 kinase inhibitor with a GM-CSF inhibitor might further reduce viral replication and hyperinflammatory responses and improve outcomes.

DCLK1 and S100A9 are expressed in epithelial compartments of the liver following acute injury and with cirrhosis and hepatocellular carcinoma. We noted strong expression of DCLK1 and S100A9 in livers of COVID-19 patients with CLD. Only weak, rare, or absent expression was noted in the livers of normal cases, or persons without significant CLD (Table 1). We found evidence for robust SARS-CoV-2 infection only in livers with CLD. The expression of ACE2 on hepatocytes in CLD likely renders this organ susceptible to infection with SARS-CoV-2. Furthermore, the results indicate that DCLK1 expression in CLD amplifies SARS-CoV-2 replication. This might worsen viremia and disease severity in patients with COVID-19 and CLD.

In addition to viral production, DCLK1 may heighten inflammatory responses through β-catenin and DCLK1/S100A9/NF-κB signaling. This involves a feed-forward loop between S100A9 and NF-κB, as DCLK1 and S100A9 each regulates the other's promoter. Furthermore, S100A9 binds TLR4 and receptors for advanced glycation end-products and could increase NF-κB signaling leading to NLRP3 activation in immune cells and release of inflammatory cytokines (e.g., IL-1β and IL-6) by immune cells. Indeed, SARS-CoV-2 infected cocultures of Calu3 and normal human PBMCs showed increased levels of p-NFκBp65^{Ser536} compared with uninfected controls (FIG. 6E). NLRP3 is activated in severe COVID-19 and may boost pro-IL-1 processing. Thus, a DCLK1/S100A9/NF-κB feed-forward cycle could help drive persistent inflammatory responses and immune dysregulation. However, in hepatoma cells, we noted the downregulation of full-length NLRP3 following viral infection (FIG. 7E, lane 4) This led to the accumulation of pro-caspase 1 and pro-IL-1β. This agrees with reports by others where the translation of NSP1 and NSP13 proteins during the early phase of SARS-CoV-2 infection inhibits NLRP3 and IL-1β secretion. Inhibition of NLRP3 and IL-1β would be expected to facilitate viral propagation during early infection and promote immune evasion.

Although an early strain of SARS-CoV-2 was used for these studies, we believe that DCLK1 and S100A9 represent host factors that would also likely be induced by more recent emerging SARS-CoV-2 variants and therefore will retain potential in the treatment of viral replication and hyperinflammation. In conclusion, the present results demonstrate the interplay between DCLK1 and SARS-CoV-2 infection in the context of CLD. DLCK1 is a host determinant that is expressed in CLD and as such represents a new target for therapy in this subset of patients with COVID-19. Small molecule inhibitors of DCLK1 kinase and S100A9/calprotectin simultaneously reduced viral production and inflammatory cytokines in vitro while impairing DCLK1 expression. Thus inhibiting DCLK1 kinase during SARS-CoV-2 infection can be used as an adjunct for the treatment of severe COVID-19.

In conclusion, and without wishing to be bound by theory, data disclosed herein provide a mechanistic explanation of severe or critical COVID-19 infection, particularly in patients with pre-existing chronic liver disease. Inhibition of DCLK1 kinase activity reduced SARS-CoV-2 virion production and hyperinflammation demonstrating a treatment protocol that can be used to reduce the severity of COVID-19 infection in patients, including, but not limited to, patients with underlying chronic liver disease.

It will be understood from the foregoing description that various modifications and changes may be made in the various embodiments of the present disclosure without departing from their true spirit. The description provided herein is intended for purposes of illustration only and is not intended to be construed in a limiting sense, except where specifically indicated. Thus, while the present disclosure has been described herein in connection with certain non-limiting embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulation of the various components and compositions described herein, the methods described herein, or in the steps or the sequence of steps of the methods described herein, without departing from the spirit and scope of the present disclosure.

### CITED REFERENCES

1. U.S Patent Application Publication 20190315753.
2. Nguyen, C. B. et al. (Z)-3,5,4′-Trimethoxystilbene Limits Hepatitis C and Cancer Pathophysiology by Blocking Microtubule Dynamics and Cell-Cycle Progression. Cancer Res 76, 4887-4896, doi:10.1158/0008-5472.CAN-15-2722 (2016).
3. Weygant, N. et al. Small molecule kinase inhibitor LRRK2-IN-1 demonstrates potent activity against colorectal and pancreatic cancer through inhibition of doublecortin-like kinase 1. Mol Cancer 13, 103, doi:10.1186/1476-4598-13-103 (2014).
4. Sureban, S. M. et al. XMD8-92 inhibits pancreatic tumor xenograft growth via a DCLK1-dependent mechanism. Cancer Lett 351, 151-161, doi:10.1016/j.canlet.2014.05.011 (2014).
5. Ali, N. et al. Hepatitis C virus-induced cancer stem cell-like signatures in cell culture and murine tumor xenografts. J Virol 85, 12292-12303, doi:10.1128/JVI.05920-11 (2011).
6. Ali, N. et al. Inflammatory and oncogenic roles of a tumor stem cell marker doublecortin-like kinase (DCLK1) in virus-induced chronic liver diseases. Oncotarget 6, 20327-20344 (2015).
7. Ali, N. et al. Doublecortin-like kinase 1 promotes hepatocyte clonogenicity and oncogenic programming via non-canonical beta-catenin-dependent mechanism. Sci Rep 10, 10578, doi:10.1038/s41598-020-67401-y (2020).
8. Olsson, A., Bjork, A., Vallon-Christersson, J., Isaacs, J. T. & Leanderson, T. Tasquinimod (ABR-215050), a quinoline-3-carboxamide anti-angiogenic agent, modulates the expression of thrombospondin-1 in human prostate tumors. Mol Cancer 9, 107, doi:10.1186/1476-4598-9-107 (2010).
9. Temesgen, Z. et al. GM-CSF Neutralization With Lenzilumab in Severe COVID-19 Pneumonia: A Case-Cohort Study. Mayo Clin Proc 95, 2382-2394, doi:10.1016/j.mayocp.2020.08.038 (2020).
10. Pang, J. et al. Efficacy and tolerability of bevacizumab in patients with severe Covid-19. Nat Commun 12, 814, doi:10.1038/s41467-021-21085-8 (2021).
11. Cavalli, G. et al. Interleukin-1 and interleukin-6 inhibition compared with standard management in patients with COVID-19 and hyperinflammation: a cohort study. Lancet Rheumatol 3, e253-e261, doi:10.1016/S2665-9913(21)00012-6 (2021).
12. U.S. Pat. No. 9,663,585.
13. U.S. Pat. No. 8,198,255.
14. U.S Patent Application Publication 20210277101.
15. U.S Patent Application Publication 20170204098.
16. U.S Patent Application Publication 20160115158.
17. U.S Patent Application Publication 20210363272
18. PCT Published Application WO 2018/222675 A1

1. A method of inhibiting SARS-CoV-2 in a host, comprising administering to the host at least one of a doublecortin-like kinase 1 (DCLK1) inhibitor and a doublecortin-like kinase 2 (DCLK2) inhibitor.

2. The method of claim 1, wherein the DCLK1 inhibitor and/or the DCLK2 inhibitor is an antibody, a small molecule, or an RNA.

3. The method of claim 1, wherein the host is a subject in need of treatment for Coronavirus infectious disease-19 (COVID-19).

4. The method of claim 3, wherein the subject also has a chronic liver disorder, disease, or condition.

5. A method of treating Coronavirus infectious disease-19 (COVID-19)-associated cytokine storm in a subject in need of such therapy, comprising administering to the subject at least one of doublecortin-like kinase 1 (DCLK1) inhibitor and a doublecortin-like kinase 2 (DCLK2) inhibitor.

6. The method of claim 5, wherein the DCLK1 inhibitor and/or the DCLK2 inhibitor is an antibody, a small molecule, or an RNA.

7. The method of claim 5, wherein the subject also has a chronic liver disorder, disease, or condition.

8. The method of claim 1, further comprising administering to the host one or more inhibitors selected from a S100 calcium binding protein A9 (S100A9) inhibitor, a S100 calcium binding protein A4 (S100A4) inhibitor, a calprotectin inhibitor, a granulocyte-macrophage colony-stimulating factor (GM-CSF) inhibitor, a Vascular endothelial growth factor (VEGF) inhibitor, and a Interleukin-6 (IL-6) inhibitor.

9-11. (canceled)

12. The method of claim 5, further comprising administering to the subject one or more inhibitors selected from a S100 calcium binding protein A9 (S100A9) inhibitor, a S100 calcium binding protein A4 (S100A4) inhibitor, a calprotectin inhibitor, a granulocyte-macrophage colony-stimulating factor (GM-CSF) inhibitor, a Vascular endothelial growth factor (VEGF) inhibitor, and a Interleukin-6 (IL-6) inhibitor.

13-15. (canceled)

16. A method of determining if a patient having coronavirus infectious disease-19 should be administered a treatment protocol for severe or critical COVID-19, comprising:
obtaining a sample of mononuclear cells from the patient and quantifying the number of DCLK1+S100A9+ mononuclear cells in the cell sample; and
administering to the patient the treatment protocol for severe or critical COVID-19 when the number of DCLK1+S100A9+ mononuclear cells in the cell sample exceeds a predetermined threshold for DCLK1+S100A9+ mononuclear cells in the cell sample by at least two-fold.

17. The method of claim 16, wherein the predetermined threshold for DCLK1+S100A9+ mononuclear cells is calculated as an average of the number of DCLK1+S100A9+ mononuclear cells measured in a population of normal subjects.

18. The method of claim 16, wherein the treatment protocol for severe or critical COVID-19 is administered when the number of DCLK1+S100A9+ mononuclear cells in the cell sample exceeds the predetermined threshold by at least four-fold.

19. The method of claim 16, wherein the DCLK1+S100A9+ mononuclear cells are further defined as DCLK1+S100A9+CD206+ mononuclear cells, and the predetermined threshold for said DCLK1+S100A9+CD206+ mononuclear cells is calculated as an average of the number of DCLK1+S100A9+CD206+ mononuclear cells measured in a population of normal subjects.

20. The method of claim 19, wherein the treatment protocol for severe or critical COVID-19 is administered when the number of DCLK1+S100A9+CD206+ mononuclear cells in the cell sample exceeds the predetermined threshold by at least four-fold.

21. The method of claim 16, wherein the patient also has a chronic liver disorder, disease, or condition.

22-23. (canceled)

24. The method of claim 16, wherein the treatment protocol, comprises administering to the subject at least one of a doublecortin-like kinase 1 (DCLK1) inhibitor and a doublecortin-like kinase 2 (DCLK2) inhibitor; and, optionally, one or more inhibitors selected from a S100 calcium binding protein A9 (S100A9) inhibitor, a S100 calcium binding protein A4 (S100A4) inhibitor, a calprotectin inhibitor, a granulocyte-macrophage colony-stimulating factor (GM-CSF) inhibitor, a Vascular endothelial growth factor (VEGF) inhibitor, and a Interleukin-6 (IL-6) inhibitor.


SEQUENCE LISTING (5 sequences)

SEQ ID NO: 1 - protein, 9 aa, Severe acute respiratory syndrome-related coronavirus 2
MSDNGPQNQ

SEQ ID NO: 2 - protein, 16 aa, Severe acute respiratory syndrome-related coronavirus 2
GFYAEGSRGGSQASSR

SEQ ID NO: 3 - protein, 8 aa, Severe acute respiratory syndrome-related coronavirus 2
NSTPGSSR

SEQ ID NO: 4 - protein, 12 aa, Severe acute respiratory syndrome-related coronavirus 2
GGSQASSRSSSR

SEQ ID NO: 5 - protein, 29 aa, Severe acute respiratory syndrome-related coronavirus 2
GSRGGSQASSRSSSRSRNSSRNSTPGSSR